(19) United States
(12) Patent Application Publication
(10) Pub. No.: US 2023/0338340 A1
(43) Pub. Date: Oct. 26, 2023

(54) Methods of Treating Cystic Fibrosis

(71) Applicant: The Trustees of Dartmouth College, Hanover, NH (US)

(72) Inventors: Nicholas P. GILL, Hanover, NH (US); Dean R. MADDEN, Hanover, NH (US); Moises CHAVEZ, Hanover, NH (US); Sahar AL-AYYOUBI, Hanover, NH (US); Patrick R. CUSHING, Hanover, NH (US); Yu ZHAO, Hanover, NH (US)

(21) Appl. No.: 18/307,522

(22) Filed: Apr. 26, 2023

Related U.S. Application Data
(63) Continuation of application No. PCT/US2021/057048, filed on Oct. 28, 2021.
(60) Provisional application No. 63/106,725, filed on Oct. 28, 2020.

(51) Int. Cl. A61K 31/4184; A61K 45/06; A61K 31/7084; A61K 31/365; A61K 31/12; A61K 31/496; A61K 31/65; A61K 31/437; A61K 31/473

(52) U.S. Cl. CPC A61K 31/4184; A61K 45/06; A61K 31/7084; A61K 31/365; A61K 31/12; A61K 31/496; A61K 31/65; A61K 31/437; A61K 31/473

(57) ABSTRACT

This disclosure relates to compounds, pharmaceutical compositions comprising them, and methods of using the compounds and compositions for treating diseases associated with interaction of proteins with PDZ domain of the CFTR-associated ligand (CAL PDZ or CALP) and/or DH domain on Disabled-2 (Dab2) protein (Dab2-DH). More particularly, this disclosure relates to methods of treating cystic fibrosis.

### CROSS-REFERENCE TO RELATED APPLICATIONS

This application claims the benefit of priority of U.S. Provisional Patent Application No. 63/106,725, filed Oct. 28, 2020, which is incorporated herein by reference in its entirety.

### STATEMENT REGARDING FEDERALLY SPONSORED RESEARCH

This invention was made with government support under Grant Nos. P20 GM113132, P30 DK117469, R01 DK101541, R01 DK104847, and, T32 GM008704, awarded by the National Institutes of Health. The government has certain rights in the invention.

### BACKGROUND OF DISCLOSURE

### Field of Disclosure

This disclosure relates to compounds, pharmaceutical compositions comprising them, and methods of using the compounds and compositions for treating diseases associated with interaction of proteins with PDZ domain of the CFTR-associated ligand (CAL PDZ or CALP) and/or DH domain on Disabled-2 (Dab2) protein (Dab2-DH). More particularly, this disclosure relates to methods of treating cystic fibrosis.

### Technical Background

Cystic fibrosis (CF) is the most common fatal recessive genetic disease among populations of European descent estimated to affect over 70000 people worldwide. Approximately 1000 new cases of CF are diagnosed each year, and more than 75% of people with CF are diagnosed by age 2. CF is caused by mutations in the gene encoding the cystic fibrosis transmembrane conductance regulator (CFTR), an ion channel required for proper fluid and ion balance in multiple epithelial tissues.

Currently, therapies for CF, such as antibiotics, anti-inflammatory agents, mucolytics, nebulized hypertonic saline, pancreatic enzyme replacement, and lung transplantation, focus on treating the symptoms of CF disease (i.e., by removing mucus and secretions from the respiratory tract, and/or reducing secondary infections associated with the disease). Even basic molecular therapies (e.g., KALYDECO® (ivacaftor), ORKAMBI® (lumacaftor/ivacaftor combination therapy), SYMDEKO® (tezacaftor/ivacaftor combination therapy), and TRIKAFTA® (elexacaftor/tezacaftor/ivacaftor combination therapy) achieve limited restoration of airway function and do not eliminate chronic bacterial infections. Therefore, there remains a need for treating the CFTR loss-of-function.

Deletion of phenylalanine 508 is the most frequent mutation causing CF and approximately 90% of CF patients are homozygous or heterozygous for the F508del mutation, which encodes a protein variant F508del-CFTR with severe loss of function. This variant exhibits impaired folding, increased degradation by endoplasmic reticulum (ER) quality control machinery, reduced capacity for Cl⁻ transport, and decreased half-life at the plasma membrane. CFTR is recycled from the cell membrane and preferentially targeted for lysosomal degradation by interaction of the CFTR C-terminus with the CFTR-associated ligand (CAL) PDZ domain (CALP; CAL PDZ). CAL has been implicated in both decreasing concentration of CFTR at the membrane and arresting CFTR trafficking in the ER, and knockdown of CAL has been shown to rescue transepithelial chloride transport in polarized CFBE41o-cells expressing F508del-CFTR by increasing the concentration of F508del-CFTR at the plasma membrane. Inhibition of the interaction between the CFTR C-terminal peptide and CALP is a potential therapeutic avenue for CF.

In addition, Disabled-2 (Dab2) is a clathrin-associated sorting protein (CLASP) that facilitates endocytosis by organizing clathrin assembly and by recruiting cargo and other adaptor proteins. Dab2 protein was identified as a key endocytic cargo adaptor for CFTR in human airway epithelial cells. Thus, regulating the CFTR-Dab2 interaction also provides a potential pharmacological approach to treating CF. Specifically, inhibition of the Dab Homology domain (DH) of Dab2 enhances CFTR abundance.

### SUMMARY OF THE DISCLOSURE

The disclosure provides a new class of compounds targeting the CFTR loss-of-function that can increase overall levels of functional protein and ameliorate the basic defect that causes CF. For example, the disclosure provides novel CALP and/or Dab2-DH inhibitors useful for treating cystic fibrosis.

Thus, one aspect of the disclosure provides a method of treating cystic fibrosis, the method including administering to a subject in need of such treatment one or more compounds as disclosed herein.

In one embodiment of the methods of the disclosure, the compounds useful in the methods of the disclosure are any one of compounds listed in Table 1:
(2R,3R)-5,7-dihydroxy-2-(3,4,5-trihydroxy-6-oxo-1-((2R,3R)-3,5,7-trihydroxychroman-2-yl)-6H-benzo[7]annulen-8-yl)chroman-3-yl 3,4,5-trihydroxybenzoate;
6-methyl-2a,2a,3,4,4a,5,6,7,8a,12b-decahydro-2H-4a¹,5-ethanofuro[4′,3′,2′:4,10]anthra[9,1-bc]oxepine-2,9,12-trione;
(1aR,1bS,aS,6aR,6bS,11aS)-3,9-dichloro-4,10-dihydroxy-1a,5a,6a,11a-tetrahydro-5H,11H-1b,6b-epoxyoxireno[2′,3′:2,3]naphtho[1,8-bc]oxireno[2′,3′:2,3]naphtho[1,8-ef]oxepine-5,11-dione;
3,4,5-trihydroxy-6-methylphthalaldehyde;
4-(4-(pyrimidin-2-yl)piperazin-1-yl)phenol;
(E)-5-chloro-9-(3-hydroxy-2-methylbutanoyl)-6a-methyl-3-(3-methylpent-1-en-1-yl)-6H-furo[2,3-h]isochromene-6,8(6aH)-dione;
1,8,9-trihydroxy-3-methoxy-6H-benzofuro[3,2-c]chromen-6-one;
N-(4-hydroxynaphthalen-1-yl)-2-oxo-2,3-dihydro-1H-benzo[d]imidazole-5-sulfonamide;
3,4′,5′-trihydroxy-5-methoxy-2′-methyl-[1,1′-biphenyl]-2-carboxylic acid;
(4S,4aR,5S,5aR,12aR)-4-(dimethylamino)-1,5,10,11,12a-pentahydroxy-6-methylene-3,12-dioxo-3,4,4a,5,5a,6,12,12a-octahydrotetracene-2-carboxamide;
5-methoxy-2-(((4-methoxy-3,5-dimethylpyridin-2-yl)methyl)sulfinyl)-1H-imidazo[4,5-b]pyridine;
(S)-6-methyl-5,6,6a,7-tetrahydro-4H-dibenzo[de, g]quinoline-10,11-diol hydrochloride;
5,5-dimethyl-5,6-dihydro-[1,2,4]triazolo[3,4-a]isoquinoline-3(2H)-thione;
methyl (1R,2R,4S)-4-(((2R,4S,5S,6S)-4-(dimethylamino)-5-hydroxy-6-methyltetrahydro-2H-pyran-2-yl)oxy)-2-ethyl-2,5,7,10-tetrahydroxy-6,11-dioxo-1,2,3,4,6,11-hexahydrotetracene-1-carboxylate;
2-chloro-13,19,23,28-tetrahydroxy-12,16,22,24,26,29-hexamethyl-13,14,19,22,23,24-hexahydro-1H-3,31-methanobenzo[e][1]azacyclononacosine-1,5,15,27,32(4H,12H,18H)-pentaone;
5,6-dihydroxy-7-isopropyl-1,1-dimethyl-1,3,4,9,10,10a-hexahydro-2H-9,4a-(epoxymethano)phenanthren-12-one;
4,5,7-trihydroxynaphthalene-1,2-dione;
(1R,4aR,12aS)-3-acetyl-1-amino-4,4a,6,7-tetrahydroxy-8,11-dimethyl-12,12a-dihydrotetracene-2,5(1H,4aH)-dione;
(E)-2-hydroxy-5-methoxy-4-((E)-3-phenylallylidene)cyclohexa-2,5-dien-1-one;
1-(3,10-dihydroxy-12-(2-(((4-hydroxyphenoxy)carbonyl)oxy)propyl)-2,6,7,11-tetramethoxy-4,9-dioxo-4,9-dihydroperylen-1-yl)propan-2-yl benzoate;
2-(2-hydroxy-4-methoxyphenyl)-2-oxo-N-phenylacetamide;
6,6′-oxybis(4-methylbenzene-1,2-diol);
3-(2,6-dihydroxy-4-methylphenoxy)-5-methylbenzene-1,2-diol;
(2R,4aS,6aS,12bR,14aS,14bR)-10-hydroxy-2,4a,6a,9,12b,14a-hexamethyl-11-oxo-1,2,3,4,4a,5,6,6a,11,12b,13,14,14a,14b-tetradecahydropicene-2-carboxylic acid;
(E)-3-(3,4-dihydroxyphenyl)-2-((3-(3,4-dihydroxyphenyl)acryloyl)oxy)propanoic acid; 5,8-dihydroxy-3-methylnaphtho[2,3-c]furan-4(9H)-one;
(E)-4-((16-methyl-2,5-dioxooxacyclohexadec-3-en-6-yl)oxy)-4-oxobutanoic acid; methyl 4a-hydroxy-7-(hydroxymethyl)-1-((3,4,5-trihydroxy-6-(hydroxymethyl)tetrahydro-2H-pyran-2-yl)oxy)-1,4a,5,7a-tetrahydrocyclopenta[c]pyran-4-carboxylate;
3,8-diamino-5-(3-(diethyl(methyl)ammonio)propyl)-6-phenylphenanthridin-5-ium bromide;
(4S,4aR,5S,5aR,6S,12aR)-4-(dimethylamino)-1,5,6,10,11,12a-hexahydroxy-6-methyl-3,12-dioxo-3,4,4a,5,5a,6,12,12a-octahydrotetracene-2-carboxamide;
(4aR,9S,10aS)-5,6-dihydroxy-7-isopropyl-1,1-dimethyl-1,3,4,9,10,10a-hexahydro-2H-9,4a-(epoxymethano)phenanthren-12-one;
4-decyl-3-methylenedihydrofuro[3,4-b]furan-2,6(3H,4H)-dione;
7-hydroxy-5-methyl-3,3a,5,11b-tetrahydro-2H-benzo[g]furo[3,2-c]isochromene-2,6,11-trione;
(1aR,1bS,5aS,6aR,6bS,11aS)-3-chloro-4,10-dihydroxy-1a,5a,6a,11a-tetrahydro-5H,11H-1b,6b-epoxyoxireno[2,3′:2,3]naphtho[1,8-bc]oxireno[2′,3′:2,3]naphtho[1,8-ef]oxepine-5,11-dione;
3-(2,5-dihydroxy-3,4-dimethoxyphenyl)butan-2-one;
   or a pharmaceutically acceptable salt thereof.

In another embodiment of the methods of the disclosure, the compounds useful in the methods of the disclosure are the compounds of formula (I):

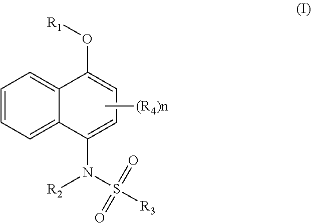

or a pharmaceutically acceptable salt thereof, wherein,
n is an integer 0, 1, or 2;
R₁ is hydrogen or C₁-C₆alkyl;
R₂ is hydrogen or C₁-C₆ alkyl;
R₃ is C₁-C₆ alkyl, aryl optionally substituted with one or more R₅, heteroaryl optionally substituted with one or more R₅, heterocyclyl optionally substituted with one or more R₅, or C₄-C₆ cycloalkyl optionally substituted with one or more R₅; and
R₄ is independently selected from halogen, —CN, —NO₂, C₁-C₆alkyl optionally substituted with one or more R₅, C₁-C₆ haloalkyl, —NH₂, —NH(C₁-C₆ alkyl), —N(C₁-C₆ alkyl)₂, —OH, —O(C₁-C₆alkyl optionally substituted with one or more R₅), C₁-C₆haloalkoxy, —CONH₂, —CONH(C₁-C₆ alkyl), —CON(C₁-C₆ alkyl)₂, —CONH—OH, —CO₂H, —CO₂(C₁-C₆ alkyl), —SH, —S(C₁-C₆ alkyl optionally substituted with one or more R₅), —SO₂R₇, —SO₂OR₇, —SO₂N(R₇)₂, aryl optionally substituted with one or more Re, heteroaryl optionally substituted with one or more Re, heterocyclyl optionally substituted with one or more R₆, and C₃-C₆ cycloalkyl optionally substituted with one or more Re;
   wherein
each R₅ is independently selected from the group consisting of halogen, —NO₂, —CN, C₁-C₆ alkyl, C₁-C₆ haloalkyl, —OH, C₁-C₆ alkoxy, C₁-C₆ haloalkoxy, —CONH₂, —CONH(C₁-C₆ alkyl), —CON(C₁-C₆ alkyl)₂, —CO₂H, —CO₂(C₁-C₆ alkyl), —SO₂R₇, —SO₂OR₇, and —SO₂N(R₇)₂;
each R₆ is independently selected from the group consisting of halogen, —NO₂, —CN, C₁-C₆ alkyl, C₁-C₆ haloalkyl, —OH, C₁-C₆ alkoxy, and C₁-C₆ haloalkoxy; and
each R₇ is independently selected from the group consisting of hydrogen, C₁-C₆ alkyl, phenyl, or tolyl.

Another aspect of the disclosure provides compounds of formula (I) as described herein, provided the compound is not:
N-(4-hydroxynaphthalen-1-yl)-2-oxo-2,3-dihydro-1H-benzo[d]imidazole-5-sulfonamide,
N-(4-hydroxynaphthalen-1-yl)-1,3-dimethyl-2-oxo-2,3-dihydro-1H-benzo[d]imidazole-5-sulfonamide,
2-((1-hydroxy-4-((2-oxo-2,3-dihydro-1H-benzo[d]imidazole)-5-sulfonamido)naphthalen-2-yl)thio)acetic acid
N-(4-hydroxynaphthalen-1-yl)benzenesulfonamide, or
N-(4-hydroxynaphthalen-1-yl)methanesulfonamide.

Another aspect of the disclosure provides a method of inhibiting protein interactions with PDZ domain of the CFTR-associated ligand (CALP), the method including administering to a subject in need of such treatment one or more compounds as disclosed herein. In certain embodiments, the disclosure provides a method of inhibiting interactions of cystic fibrosis transmembrane conductance regulator (CFTR) with CALP, the method including administering to a subject in need of such treatment one or more compounds as disclosed herein.

Another aspect of the disclosure provides a method of inhibiting protein interactions with DH domain of Dab2 protein (e.g., Dab2-DH), the method including administering to a subject in need of such treatment one or more compounds as disclosed herein.

### BRIEF DESCRIPTION OF THE DRAWINGS

The accompanying drawings are included to provide a further understanding of the compositions and methods of the disclosure, and are incorporated in and constitute a part of this specification. The drawings illustrate one or more embodiment(s) of the disclosure and, together with the description, serve to explain the principles and operation of the disclosure.

FIG. 1 shows CALP inhibition dose-response of several of the compounds of the disclosure.

FIG. 2 displays graphs showing the effect of compounds according to example embodiments on the cytotoxicity and anti-proliferation of wild-type CF bronchial epithelial (CFBE) cells.

FIG. 3 displays a graph showing the results of a functionary assay to determine CFTR rescue in CFBE cells when treated with a compound according to an example embodiment.

FIG. 4 displays immunoblots of input, supernatant, and pull-down fractions following detection with an anti-Dab2 antibody according to example embodiments.

FIG. 5 shows various views of the crystal structure determined of the Dab2-DH:STA02 complex according to an example embodiment.

FIG. 6 displays graphs of the toxicity and anti-proliferative effects of compounds according to example embodiments. iD01 is 2-hydroxyestradiol: iD03 is dipyridamole.

FIG. 7 displays a graph of the observed increased abundance of CFTR when WVT-CFBE cells were treated with a compound according to an example embodiment.

FIG. 8 displays a graph showing the changes in ASL volume upon addition of compounds according to example embodiments.

### DETAILED DESCRIPTION

Before the disclosed processes and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

In view of the present disclosure, the methods and compositions described herein can be configured by the person of ordinary skill in the art to meet the desired need. In general, the disclosed materials and methods provide improvements in treatment of diseases or disorders associated with interactions mediated by the CAL PDZ or Dab2 DH domains.

Thus, in one aspect, the disclosure provides a method of treating cystic fibrosis, the method including administering to a subject in need of such treatment one or more compounds as disclosed herein.

In certain embodiments, the compound used in the methods of the disclosure as described herein is any one of Examples 1-35 disclosed in Table 1. In certain embodiments, the compound used in the methods of the disclosure is any one of Examples 1-13 disclosed in Table 1. In certain embodiments, the compound used in the methods of the disclosure is any one of Examples 14-35 disclosed in Table 1.

In certain embodiments, the compound used in the methods of the disclosure as described herein is (E)-5-chloro-9-(3-hydroxy-2-methylbutanoyl)-6a-methyl-3-(3-methylpent-1-en-1-yl)-6H-furo[2,3-h]isochromene-6,8(6aH)-dione (example 6).

In certain embodiments, the compound used in the methods of the disclosure as described herein is 3,4′,5′-trihydroxy-5-methoxy-2′-methyl-[1,1′-biphenyl]-2-carboxylic acid (example 9).

In certain embodiments, the compound used in the methods of the disclosure as described herein is (4S,4aR,5S,5aR,12aR)-4-(dimethylamino)-1,5,10,11,12a-pentahydroxy-6-methylene-3,12-dioxo-3,4,4a,5,5a,6,12,12a-octahydrotetracene-2-carboxamide (example 10).

In certain embodiments, the compound used in the methods of the disclosure as described herein is (S)-6-methyl-5,6,6a,7-tetrahydro-4H-dibenzo[de,g]quinoline-10,11-diol hydrochloride (example 12).

In certain embodiments, the compound used in the methods of the disclosure as described herein is 5,5-dimethyl-5,6-dihydro-[1,2,4]triazolo[3,4-a]isoquinoline-3(2H)-thione (example 13).

In certain embodiments, the compound used in the methods of the disclosure as described herein is of formula (I):

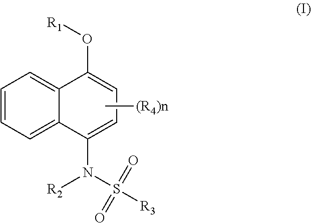

or a pharmaceutically acceptable salt thereof, wherein n, R₁, R₂, R₃, and R₄ are as described above.

In certain embodiments, in the compound of formula (I) as described herein, R₁ is hydrogen or C₁-C₃ alkyl (e.g., methyl). In certain other embodiments, R₁ is hydrogen. In certain other embodiments, R₁ is C₁-C₃ alkyl, such as methyl.

In certain embodiments, in the compound of formula (I) as described herein, R₂ is hydrogen or C₁-C₃ alkyl (e.g., methyl). In certain other embodiments, R₂ is hydrogen. In certain other embodiments, R₂ is C₁-C₃ alkyl, such as methyl.

In certain embodiments, in the compound of formula (I) both R₁ and R₂ are independently hydrogen, i.e., the compounds have the formula:

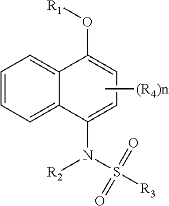

In certain embodiments, in the compound of formula (I) as described herein, R₃ is C₁-C₆ alkyl. In certain other embodiments, R₃ is C₁-C₃ alkyl. In certain other embodiments, R₃ is as methyl.

In certain embodiments, in the compound of formula (I) as described herein, R₃ is aryl optionally substituted with one or more R₅, heteroaryl optionally substituted with one or more R₅, heterocyclyl optionally substituted with one or more R₅, or C₄-C₈cycloalkyl optionally substituted with one or more R₅. In certain other embodiments, R₃ is aryl (e.g., phenyl or naphthyl) optionally substituted with one or more R₅. In certain other embodiments, R₃ is heteroaryl optionally substituted with one or more R₅ or heterocyclyl optionally substituted with one or more R₅. In certain other embodiments, R₃ is heteroaryl optionally substituted with one or more R₅. For example, in certain embodiments, R₃ is an optionally substituted bicyclic heteroaryl. In certain embodiments, R₃ is an optionally substituted benzimidazolyl, benzofuranyl, benzoxadiazolyl, or benzoxazolyl. In other embodiments, R₃ is an optionally substituted benzimidazolyl (e.g., 2-oxo-2,3-dihydro-1H-benzo[d]imidazolyl).

In certain embodiments, in the compound of formula (I) as described herein, R₃ is unsubstituted benzimidazolyl (e.g., 2-oxo-2,3-dihydro-1H-benzo[d]imidazolyl).

In the compound of formula (I) as otherwise described herein, each R₅ when present is halogen, C₁-C₆ alkyl, C₁-C₆haloalkyl, —OH, or C₁-C₆ alkoxy. For example, each R₅ is halogen, C₁-C₃ alkyl, C₁-C₃ haloalkyl, —OH, or C₁-C₃alkoxy. In certain embodiments, each R₅ is halogen or C₁-C₃ alkyl (e.g., methyl).

As noted above, the naphthyl moiety in the compounds of formula (I) may be optionally substituted. In certain embodiments, the naphthyl moiety in the compounds of formula (I) is unsubstituted, i.e., n is 0. In certain embodiments, the naphthyl moiety in the compounds of formula (I) is substituted. For example, in certain embodiments, n is 1 or 2. In certain embodiments, n is 1.

Each R₄ in the compound of formula (I) as otherwise described herein independently may be selected from halogen, C₁-C₆ alkyl optionally substituted with one or more R₅, C₁-C₆haloalkyl, —NH₂, —NH(C₁-C₆ alkyl), —N(C₁-C₆ alkyl)₂, —OH, —O(C₁-C₆ alkyl optionally substituted with one or more R₅), C₁-C₆ haloalkoxy, —CONH₂, —CONH(C₁-C₆ alkyl), —CON(C₁-C₆ alkyl)₂, —CO₂H, —CO₂(C₁-C₆ alkyl), —SH, and —S(C₁-C₆ alkyl optionally substituted with one or more R). In certain embodiments, each R₄ is independently selected from —NH₂, —NH(C₁-C₆ alkyl), —N(C₁-C₆ alkyl)₂, —OH, —O(C₁-C₆ alkyl optionally substituted with one or more R₅), C₁-C₆haloalkoxy, —CONH₂, —CONH(C₁-C₆alkyl), —CON(C₁-C₆alkyl)₂, —CO₂H, —CO₂(C₁-C₆ alkyl), —SH, and —S(C₁-C₆ alkyl optionally substituted with one or more R₅). In certain embodiments, each R₄ is independently selected from —OH, —O(C₁-C₆ alkyl optionally substituted with one or more R₅), C₁-C₆ haloalkoxy, —SH, and —S(C₁-C₆ alkyl optionally substituted with one or more R₅). In certain embodiments, each R₄ is independently selected from —OH, —O(C₁-C₃ alkyl optionally substituted with one or more R₅), C₁-C₆haloalkoxy, —SH, and —S(C₁-C₃ alkyl optionally substituted with one or more R₅). In certain other embodiments, each R₄ is independently selected from —OH, —O(C₁-C₃ alkyl optionally substituted with one or more R₅), and C₁-C₃haloalkoxy. In certain other embodiments, each R₄ is independently selected from —SH and —S(C₁-C₃alkyl optionally substituted with one or more R). In certain embodiments, each R₄ is independently selected from halogen, —OH, —O(C₁-C₃ alkyl optionally substituted with one or more R₅), C₁-C₃haloalkoxy, —SH, and —S(C₁-C₃alkyl optionally substituted with one or more R₅). In certain other embodiments, one R₄ is selected from —OH, —O(C₁-C₃ alkyl optionally substituted with one or more R₅), and C₁-C₃haloalkoxy, and the other R₄ if present is halogen. In certain other embodiments, one R₄ is —SH or —S(C₁-C₃ alkyl optionally substituted with one or more R₅), and the other R₄ if present is halogen. In certain embodiments, when R₄ group is substituted with R₅, R₅ is selected from —OH, C₁-C₆alkoxy, —CO₂H, and —CO₂(C₁-C₆ alkyl).

In some embodiments of the methods of the disclosure, at least a 5% (e.g., at least 10%, at least 15%, at least 20%, at least 25%, at least 30%, at least 35%, at least 40%, at least 50%, at least 60%, at least 70%) improvement in one or more symptoms of the disease or disorder of the disclosure as described herein is sufficient to classify the subject as responding to the method of treatment.

The compounds and compositions of the disclosure as described herein may also be administered in combination with one or more secondary therapeutic agents. Thus, in certain embodiment, the method also includes administering to a subject in need of such treatment an effective amount of one or more compounds of the disclosure as described herein (i.e., compounds of Table 1, Table 2, and/or formula (I)) or a pharmaceutical composition of the disclosure as described herein and one or more secondary therapeutic agents.

In certain embodiments, the secondary therapeutic agent is a CFTR modulator. Examples of CFTR modulators include, but are not limited to, ivacaftor (sold as Kalydeco®, Vertex Pharmaceuticals, Boston, MA, USA), lumacaftor, tezacaftor, and their combinations, such as lumacaftor/ivacaftor (sold as Orkambi®, Vertex Pharmaceuticals, Boston, MA, USA), tezacaftor/ivacaftor (sold as Symdeko®, Vertex Pharmaceuticals), and elexacaftor/tezacaftor/ivacaftor (sold as Trikafta™, Vertex Pharmaceuticals).

In certain embodiments, the secondary therapeutic agent is selected from antibiotics, anti-inflammatory agents, mucoactive agents (such as mucolytics and nebulized hypertonic saline), and combinations thereof. In some embodiments, the secondary therapeutic agent is a mucolytic agent. Examples of mucolytic agents include, but are not limited to, dornase alfa (sold as Pulmozyme®, Genentech USA, Inc., South San Francisco, CA, USA), denufosol, acetylcysteine, ambroxol, bromhexine, carbocisteine, erdosteine, hypertonic saline, etc. In some embodiments, the secondary therapeutic agent is an antibiotic. Examples of antibiotics include, but are not limited to, penicillins, cephalosporins, carbapenems, sulfas, tetracyclines, vancomycin, lincosamides, oxazolidinone, aminoglycosides, macrolides, quinolones, aztreonam, colistimethate/Colistin®, rifamycin, clofazimine, ethambutol, and the like.

When administered as a combination, the compounds and compositions of the disclosure as described herein and the secondary therapeutic agents can be formulated as separate compositions that are given simultaneously or sequentially, or the therapeutic agents can be given as a single composition. In certain embodiments, the secondary therapeutic agent may be administered in an amount below its established half maximal inhibitory concentration (IC₅₀). For example, the secondary therapeutic agent may be administered in an amount less than 1% of, e.g., less than 10%, or less than 25%, or less than 50%, or less than 75%, or even less than 90% of the inhibitory concentration (IC₅₀).

The compounds of the disclosure may be administered as a pharmaceutical composition. Thus, in some embodiments of the methods of the disclosure, the compound is administered as a pharmaceutical composition comprising the compound and a pharmaceutically acceptable carrier, solvent, adjuvant or diluent. The exact nature of the carrier, solvent, adjuvant, or diluent will depend upon the desired use for the composition, and may range from being suitable or acceptable for veterinary uses to being suitable or acceptable for human use.

When used to treat or prevent such diseases, the compounds described herein may be administered singly, as mixtures of one or more compounds or in mixture or combination with other agents useful for treating such diseases and/or the symptoms associated with such diseases.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate): lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC₅₀ of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

### Definitions

The following terms and expressions used herein have the indicated meanings.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

Terms used herein may be preceded and/or followed by a single dash, “-”, or a double dash, “=”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” (i.e., the attachment is via the last portion of the name) unless a dash indicates otherwise. For example, C₁-C₆alkoxycarbonyloxy and —OC(O)C₁-C_{C}alkyl indicate the same functionality; similarly arylalkyl and -alkylaryl indicate the same functionality.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkyl” as used herein, means a straight or branched chain hydrocarbon containing from 1 to 10 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂—, —CH₂CH₂CHC(CH₃)—, and —CH₂CH(CH₂CH₃)CH₂—.

The term “aryl,” as used herein, means a phenyl (i.e., monocyclic aryl), or a bicyclic ring system containing at least one phenyl ring or an aromatic bicyclic ring containing only carbon atoms in the aromatic bicyclic ring system. The bicyclic aryl can be azulenyl, naphthyl, or a phenyl fused to a monocyclic cycloalkyl, a monocyclic cycloalkenyl, or a monocyclic heterocyclyl. The bicyclic aryl is attached to the parent molecular moiety through any carbon atom contained within the phenyl portion of the bicyclic system, or any carbon atom with the napthyl or azulenyl ring. The fused monocyclic cycloalkyl or monocyclic heterocyclyl portions of the bicyclic aryl are optionally substituted with one or two oxo and/or thioxo groups. Representative examples of the bicyclic aryls include, but are not limited to, azulenyl, naphthyl, dihydroinden-1-yl, dihydroinden-2-yl, dihydroinden-3-yl, dihydroinden-4-yl, 2,3-dihydroindol-4-yl, 2,3-dihydroindol-5-yl, 2,3-dihydroindol-6-yl, 2,3-dihydroindol-7-yl, inden-1-yl, inden-2-yl, inden-3-yl, inden-4-yl, dihydronaphthalen-2-yl, dihydronaphthalen-3-yl, dihydronaphthalen-4-yl, dihydronaphthalen-1-yl, 5,6,7,8-tetrahydronaphthalen-1-yl, 5,6,7,8-tetrahydronaphthalen-2-yl, 2,3-dihydrobenzofuran-4-yl, 2,3-dihydrobenzofuran-5-yl, 2,3-dihydrobenzofuran-6-yl, 2,3-dihydrobenzofuran-7-yl, benzo[d][1,3]dioxol-4-yl, benzo[d][1,3]dioxol-5-yl, 2H-chromen-2-on-5-yl, 2H-chromen-2-on-6-yl, 2H-chromen-2-on-7-yl, 21H-chromen-2-on-8-yl, isoindoline-1,3-dion-4-yl, isoindoline-1,3-dion-5-yl, inden-1-on-4-yl, inden-1-on-5-yl, inden-1-on-6-yl, inden-1-on-7-yl, 2,3-dihydrobenzo[b][1,4]dioxan-5-yl, 2,3-dihydrobenzo[b][1,4]dioxan-6-yl, 2H-benzo[b][1,4]oxazin3(4H)-on-5-yl, 2H-benzo[b][1,4]oxazin3(4H)-on-6-yl, 2H-benzo[b][1,4]oxazin3(4H)-on-7-yl, 2H-benzo[b][1,4]oxazin3(4H)-on-8-yl, benzo[d]oxazin-2(3H)-on-5-yl, benzo[d]oxazin-2(3H)-on-6-yl, benzo[d]oxazin-2(3H)-on-7-yl, benzo[d]oxazin-2(3H)-on-8-yl, quinazolin-4(3H)-on-5-yl, quinazolin-4(3H)-on-6-yl, quinazolin-4(3H)-on-7-yl, quinazolin-4(3H)-on-8-yl, quinoxalin-2(1H)-on-5-yl, quinoxalin-2(1H)-on-6-yl, quinoxalin-2(1H)-on-7-yl, quinoxalin-2(1H)-on-8-yl, benzo[d]thiazol-2(3H)-on-4-yl, benzo[d]thiazol-2(3H)-on-5-yl, benzo[d]thiazol-2(3H)-on-6-yl, and, benzo[d]thiazol-2(3H)-on-7-yl. In certain embodiments, the aryl is a phenyl or a naphthyl ring. In certain embodiments, the aryl is a phenyl ring.

The term “cycloalkyl” as used herein, means a monocyclic or a bicyclic cycloalkyl ring system. Monocyclic ring systems are cyclic hydrocarbon groups containing from 3 to 8 carbon atoms, where such groups can be saturated or unsaturated, but not aromatic. In certain embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. The bridged or fused bicyclic cycloalkyl is attached to the parent molecular moiety through any carbon atom contained within the monocyclic cycloalkyl ring. Cycloalkyl groups are optionally substituted with one or two groups which are independently oxo or thioxo. In certain embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thioxo. In certain embodiments, cycloalkyl is a monocyclic ring system containing from 3 to 8 carbon atoms.

The term “halo” or “halogen” as used herein, means —Cl, —Br, —I or —F.

The terms “haloalkyl” and “haloalkoxy” refer to an alkyl or alkoxy group, as the case may be, which is substituted with one or more halogen atoms.

The term “heteroaryl,” as used herein, means a monocyclic heteroaryl or a bicyclic ring system containing at least one heteroaromatic ring. The monocyclic heteroaryl can be a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, a monocyclic cycloalkyl, a monocyclic cycloalkenyl, a monocyclic heterocyclyl, or a monocyclic heteroaryl. The fused cycloalkyl or heterocyclyl portion of the bicyclic heteroaryl group is optionally substituted with one or two groups which are independently oxo or thioxo. When the bicyclic heteroaryl contains a fused cycloalkyl, cycloalkenyl, or heterocyclyl ring, then the bicyclic heteroaryl group is connected to the parent molecular moiety through any carbon or nitrogen atom contained within the monocyclic heteroaryl portion of the bicyclic ring system. When the bicyclic heteroaryl is a monocyclic heteroaryl fused to a benzo ring, then the bicyclic heteroaryl group is connected to the parent molecular moiety through any carbon atom or nitrogen atom within the bicyclic ring system. Representative examples of bicyclic heteroaryl include, but are not limited to, benzimidazolyl, benzofuranyl, benzothienyl, benzoxadiazolyl, benzoxazolyl, benzoxathiadiazolyl, benzothiazolyl, cinnolinyl, 5,6-dihydroquinolin-2-yl, 5,6-dihydroisoquinolin-1-yl, furopyridinyl, indazolyl, indolyl, isoquinolinyl, naphthyridinyl, quinolinyl, purinyl, 5,6,7,8-tetrahydroquinolin-2-yl, 5,6,7,8-tetrahydroquinolin-3-yl, 5,6,7,8-tetrahydroquinolin-4-yl, 5,6,7,8-tetrahydroisoquinolin-1-yl, thienopyridinyl, 4,5,6,7-tetrahydrobenzo[c][1,2,5]oxadiazolyl, 2,3-dihydrothieno[3,4-b][1,4]dioxan-5-yl, and 6,7-dihydrobenzo[c][1,2,5]oxadiazol-4(5H)-onyl. In certain embodiments, the fused bicyclic heteroaryl is a 5 or 6 membered monocyclic heteroaryl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused cycloalkyl, cycloalkenyl, and heterocyclyl groups are optionally substituted with one or two groups which are independently oxo or thioxo.

The terms “heterocyclyl” as used herein, mean a monocyclic heterocycle or a bicyclic heterocycle. The monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The monocyclic heterocycle is connected to the parent molecular moiety through any carbon atom or any nitrogen atom contained within the monocyclic heterocycle. Representative examples of monocyclic heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to either a phenyl, a monocyclic cycloalkyl, a monocyclic cycloalkenyl, a monocyclic heterocycle, or a monocyclic heteroaryl. The bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. Heterocyclyl groups are optionally substituted with one or two groups which are independently oxo or thioxo. In certain embodiments, the bicyclic heterocyclyl is a 5 or 6 membered monocyclic heterocyclyl ring fused to phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thioxo.

The term “oxo” as used herein means a ═O group.

The term “substituted”, as used herein, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which can be replaced with the radical of a suitable substituent.

The phrase “one or more” substituents, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different. As used herein, the term “independently selected” means that the same or different values may be selected for multiple instances of a given variable in a single compound.

It will be apparent to one skilled in the art that certain compounds of this disclosure may exist in tautomeric forms, all such tautomeric forms of the compounds being within the scope of the disclosure. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Both the R and the S stereochemical isomers, as well as all mixtures thereof, are included within the scope of the disclosure.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

“Pharmaceutically acceptable salt” refers to both acid and base addition salts.

“Therapeutically effective amount” refers to that amount of a compound which, when administered to a subject, is sufficient to effect treatment for a disease or disorder described herein. The amount of a compound which constitutes a “therapeutically effective amount” will vary depending on the compound, the disorder and its severity, and the age of the subject to be treated, but can be determined routinely by one of ordinary skill in the art.

“Treating” or “treatment” as used herein covers the treatment of a disease or disorder described herein, in a subject, preferably a human, and includes:
i. inhibiting a disease or disorder, i.e., arresting its development;
ii. relieving a disease or disorder, i.e., causing regression of the disorder;
iii. slowing progression of the disorder; and/or
iv. inhibiting, relieving, ameliorating, or slowing progression of one or more symptoms of the disease or disorder.
   In certain embodiments, treating as used herein includes inhibiting a disease or disorder. In certain embodiments, treating as used herein includes inhibiting, relieving, ameliorating, or slowing progression of one or more symptoms of the disease or disorder.

“Subject” or “patient” refers to a warm blooded animal such as a mammal, preferably a human, or a human child, which is afflicted with, or has the potential to be afflicted with one or more diseases and disorders described herein.

### Methods of Preparation

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Ed., Wiley-Interscience, 2001; or Vogel, A Textbook of Practical Organic Chemistry, Including Qualitative Organic Analysis, Fourth Ed., New York: Longman, 1978).

Compounds as described herein can be purified by any of the means known in the art, including chromatographic means, such as HPLC, preparative thin layer chromatography, flash column chromatography and ion exchange chromatography. Any suitable stationary phase can be used, including normal and reversed phases as well as ionic resins. Most typically the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, ed F. Stahl, Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry,” Plenum Press, London and New York 1973, in T. W. Greene and P. G. M. Wuts, “Protective Groups in Organic Synthesis,” Third edition, Wiley, New York 1999, in “The Peptides”; Volume 3 (editors: E. Gross and J. Meienhofer), Academic Press, London and New York 1981, in “Methoden der organischen Chemie,” Houben-Weyl, 4.sup.th edition, Vol. 15/1, Georg Thieme Verlag, Stuttgart 1974, in H.-D. Jakubke and H. Jescheit, “Aminosauren, Peptide, Proteine,” Verlag Chemie, Weinheim, Deerfield Beach, and Basel 1982, and/or in Jochen Lehmann, “Chemie der Kohlenhydrate: Monosaccharide and Derivate,” Georg Thieme Verlag, Stuttgart 1974. The protecting groups may be removed at a convenient subsequent stage using methods known from the art.

The compounds disclosed herein can be made using procedures familiar to the person of ordinary skill in the art and as described herein. For example, compounds of structural formula (I) can be prepared according to known methods or they can be purchased from commercial sources.

### Examples

The methods of the disclosure are illustrated further by the following examples, which are not to be construed as limiting the disclosure in scope or spirit to the specific procedures and compounds described in them.

### Examples 1-35: CALP and Dab2 Inhibition Assay

CALP and/or Dab2 inhibition assays were performed in biochemical “mix-and-measure” format in order to evaluate the effects on activation and inhibition of CALP and/or Dab2 by the compounds of the disclosure. Brief assay procedure is provided below. Unless otherwise specified, all of the compounds were obtained from commercial sources.

CALP inhibitors were identified by fluorescence polarization (FP) competition assays in high-throughput screening (HTS) format. Recombinant CAL PDZ was expressed and purified in E. coli as previously described (Amacher et al., Acta Cyst. (2011) F67, 600-603), and dialyzed into FP storage buffer (25 mM Tris pH 8.5, 150 mM NaCl, 0.1 mM TCEP, and 0.02% sodium azide). A fluorescent reporter peptide, NPG32, was purchased commercially with the following sequence: F*-ANSRWQTSII (F* denotes a N-terminal fluorescein group coupled via an aminohexanoic acid linker). Initial single-dose HTS was performed with libraries comprising ˜52,000 compounds (FAST Lab—Novartis Institutes for BioMedical Research). First, 25 nL of 5 mM small-molecule was transferred to 1536-well Grenier plates. 3 μL of 3.708 μM CALP in FP buffer (FP storage buffer supplemented with 0.1 mM IgG and 30 μM thesis) was added, and plates were incubated for 10 min. Next, 3 μL of 60 nM NPG32 (in FP buffer) was added. Final reaction volumes were 6 μL and contained 1.854 μM CALP, 30 nM NPG32, and 20.83 μM small-molecule. Negative controls contained equivalent amounts of DMSO (0.417%), while positive controls contained 10 μM NPG35 (ANSRWQVTR[Tle] where [Tle] is L-tert leucine), an unlabeled, high-affinity peptide inhibitor of CALP (K_{d}=<1 μM). Plates were incubated for 30 minutes at room temperature and anisotropy was measured (480 nm excitation wavelength and 535 nm emission wavelength). 126 compounds showed >15% inhibition (3×SD) and were subjected to subsequent validation. Compounds were validated in triplicate using 8-point dilution series in half-log increments with two orthogonal assays: FRET and TMR-based FP assay. For the FRET assay, a cerulean-CALP fusion protein (Cer-CALP) was expressed and purified as previously described (Zhao et al., 2018). A fluorescent reporter peptide, PRC34, was purchased commercially with the following sequence: TMR*-ANSRWPTSII (TMR* denotes a N-terminal tetramethylrhodamine group coupled via an aminohexanoic acid linker). First, 40 nL of compound was transferred to the plates. 5 μL of 16.632 μM Cer-CALP was added, and plates were incubated for 30 min. Next, 5 μL of 16.632 μM PRC34 was added, and plates were incubated for 30 min. Final reaction volumes were 10 μL and contained 8.316 μM CALP, 8.316 μM PRC36, and variable concentrations of small-molecule. Negative controls contained equivalent amounts of DMSO (0.4%), while positive controls contained 100 μM NPG30 (sequence: ANSRWPVTRV). Plates were incubated for 30 min, and emission at 475 nm and 575 nm was measured following excitation at 430 nm. TMR FP assays were conducted as described for the FITC-based assay with the following exceptions: TMR was used as the reporter peptide, the final CALP concentration was 11.952 μM, and the positive control used was 100 μM NPG30.

Dab-2 inhibitors were also identified by FP competition assays via HTS. Human-Dab2-DH, residues 31-191 (Uniprot: P98082), and trDab2-DH (residues 33-178) was subcloned into the pET16b vector via PCR utilizing the restriction enzyme sites of Xhol and BamHI. The construct was designed with a 3C protease cleavage site directly upstream and in-frame with the protein coding sequence. DNA was sequence verified. (0.1 mM) IPTG was used to induce protein expression in E. coli BL21(DE3)RIL cells, after reaching an O.D. of 06. Cells were grown overnight at 20° C., after which they were lysed, and the protein purified through a nickel affinity column (HisTrap HP, GE Healthcare Life Sciences; product code:17-5247-01) and size-exclusion chromatography (SEC) using a HiLoad Superdex 200 PG (GE Healthcare Life Sciences; product code:28989336). Followed by 3C cleavage of the 10×His tag (unless otherwise indicated), and a second nickel affinity column to remove the 3C protease and excess tag. Dab2-DH was dialyzed into FP storage buffer (25 mM Tris pH 8.0, 150 mM NaCl, 0.1 mM TCEP, and 0.02% sodium azide). Fluorescent reporter peptides, TIVR*STA02 (TMR*-QNGFDNPNYQPQENMQA) and TMR*STA03 (TMR*-QNGFDNPNYQPQ), were purchased commercially with the following sequence where TMR* denotes a N-terminal tetramethylrhodamine group coupled via an aminohexanoic acid linker. An unlabled positive control peptide STA02 (QNGFDNPNYQPQENMQA) was also purchased commercially. Inhibitors of Dab2-DH were identified from a variety of sources with various protocols outlined below.

For screens conducted at the Novartis Institutes for BioMedical Research FAST Lab, single-dose HTS assays were performed with libraries comprising ˜52,000 compounds at 20 μM doses. Experiments were conducted as described for screens at the University of Pittsburgh Lab and Drug Discovery Institute, except that Dab2-DH, not trDab2-DH, was used as the protein source. 377 compounds exhibited >29% inhibitory activity (3×SD) and were subjected to dose-response validation assays.

For screens conducted at the Broad Institute, compound transfers were done using a 384-well pin tool equipped with 100 nL hydrophobic surface coated pins. 100 nL of DMSO (negative control), STA02 (positive control), or library compounds were dispensed into 20 μL of protein (200 nM) and reporter (F*-STA02, 30 nM) mixture with 0.1 mg/ml mouse IgG and 0.5 mM Thesit. Final concentrations in the mixture were DMSO (1%), STA02 (20 μM), and compounds (18 μM). Plates were incubated for 35-45 minutes before measuring polarization. Compounds were tested in duplicates on two separate plates. Positive and negative controls were included in each individual plate.

For experiments conducted at the University of Pittsburgh Lab and Drug Discovery Institute, single-dose HTS assays were performed with a library containing ˜220,000 compounds. On the day of screening, a master mix of equal parts trDab2-DH (900 nM) and TMP-STA03 (90 nM) was prepared. Compound plates containing assay ready 2 μL aliquots of a 1 mM stock solution in DMSO were thawed, reconstituted in FP buffer to a concentration of 30 μM in 3% DMSO, and five μL transferred to low volume assay plates using a Perkin Elmer MDT. Ten μL of master mix were added by a Microflo bulk liquid dispenser (Biotek, Winooski, VT) and processed as described in the general protocol above. Each plate contained 32 wells of positive (Dab2-DH+probe+DMSO) and negative (Dab2-DH+probe +10 μM STA03) controls. Plates were read at 1.5-h and 4-h on the Envision multilabel reader.

The results of CALP and/or Dab2 inhibition assay for the representative compounds of the disclosure are provided in Table 1.

TABLE 1

| | | | Inhibitory activity | | | |
|---|---|---|---|---|---|---|
| | | | CALP | | Dab2 | |
| | | | IC₅₀ | at 100 | IC₅₀ | at |
| Ex. | Structure | Name | (μM) | μM | (μM) | Cₘₐₓ |
| 1 | 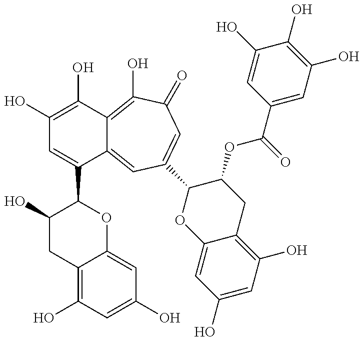 [SMILES: O=C(O[C@@H]1Cc2c(O)cc(O)cc2O[C@@H]1c1cc(=O)c(O)c2c(O)c(O)cc([C@H]3Oc4cc(O)cc(O)c4C[C@H]3O)c2c1)c1cc(O)c(O)c(O)c1] | (2R,3R)-5,7-dihydroxy- 2-(3,4,5-trihydroxy-6- oxo-1-((2R,3R)-3,5,7- trihydroxychroman-2- yl)-6H- benzo[7]annulen-8- yl)chroman-3-yl 3,4,5- trihydroxybenzoate (theaflavin-3- gallate(TF2A); CAS RN 30462-34-1; PubChem CID 169167) | 11.32 | −65.53 | 2.45 | −79.61 |
| 2 | 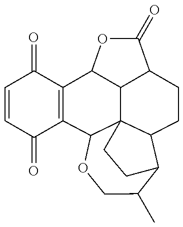 [SMILES: CC1COC2C3=C(C(=O)C=CC3=O)C3OC(=O)C4CCC5C1CCC25C43] | 6-methyl- 2a,2a¹,3,4,4a,5,6,7,8a, 12b-decahydro-2H- 4a¹,5- ethanofuro[4′,3′,2′:4,10] anthra[9,1-bc]oxepine- 2,9,12-trione (PubChem CID 101110) | 1.30 | −67.12 | 0.22 | −89.99 |
| 3 | 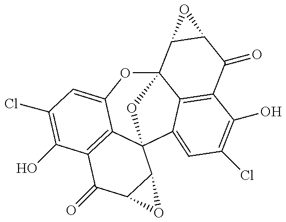 [SMILES: O=C1c2c(O)c(Cl)cc3c2[C@@]2(Oc4cc(Cl)c(O)c5c4[C@]3(O2)[C@@H]2O[C@@H]2C5=O)[C@@H]2O[C@H]12] | (1aR,1bS,5aS,6aR,6bS, 11aS)-3,9-dichloro- 4,10-dihydroxy- 1a,5a,6a,11a- tetrahydro-5H,11H- 1b,6b- epoxyoxireno[2′,3′:2,3] naphtho[1,8- bc]oxireno[2′,3′:2,3]naph- tho[1,8-ef]oxepine- 5,11-dione (spiroxin B; PubChem CID 10366321) | 25.32 | −80.85 | 0.26 | −93.47 |
| 4 | 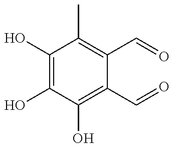 | 3,4,5-trihydroxy-6- methylphthalaldehyde (flavipin; CAS RN 483- 53-4; PubChem CID 3083587) | 10.24 | −94.09 | 3.42 | −85.95 |
| 5 | 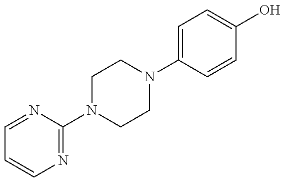 [SMILES: Oc1ccc(N2CCN(c3ncccn3)CC2)cc1] | 4-(4-(pyrimidin-2- yl)piperazin-1- yl)phenol (PubChem CID 2100296) | 40.52 | −46.33 | 20.61 | −44.86 |
| 6 | 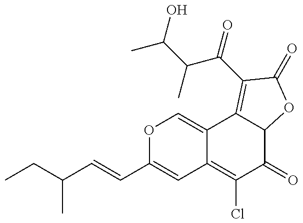 [SMILES: CCC(C)/C=C/C1=CC2=C(Cl)C(=O)C3OC(=O)C(C(=O)C(C)C(C)O)=C3C2=CO1] | (E)-5-chloro-9-(3- hydroxy-2- methylbutanoyl)-6a- methyl-3-(3- methylpent-1-en-1-yl)- 6H-furo[2,3- h]isochromene- 6,8(6aH)-dione (PubChem CID 74075505) | 34.21 | −72.96 | 7.54 | −90.85 |
| 7 | 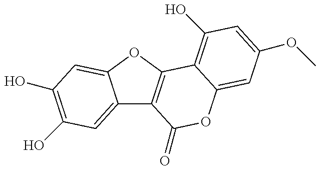 [SMILES: COc1cc(O)c2c(c1)oc(=O)c1c3cc(O)c(O)cc3oc21] | 1,8,9-trihydroxy-3- methoxy-6H- benzofuro[3,2- c]chromen-6-one (wedelolactone; CAS RN 524-12-9; PubChem CID 5281813) | 1.24 | −74.42 | | |
| 8 | 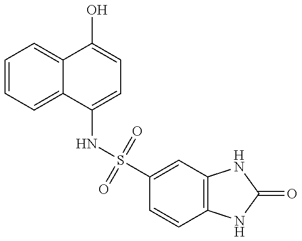 [SMILES: O=c1[nH]c2ccc(S(=O)(=O)Nc3ccc(O)c4ccccc34)cc2[nH]1] | N-(4- hydroxynaphthalen-1- yl)-2-oxo-2,3-dihydro- 1H-benzo[d]imidazole- 5-sulfonamide (PubChem CID 2133822) | 4.57 | −72.16 | | |
| 9 | 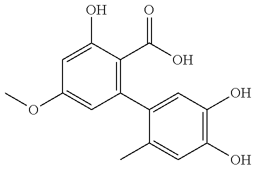 [SMILES: COc1cc(O)c(C(=O)O)c(-c2cc(O)c(O)cc2C)c1] | 3,4′,5′-trihydroxy-5- methoxy-2′-methyl- [1,1′-biphenyl]-2- carboxylic acid (alutenusin; CAS RN 31186-12-6; PubChem CID 6918469) | | | 0.037 | −88.07 |
| 10 | 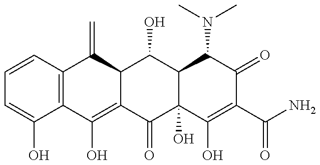 [SMILES: C=C1c2cccc(O)c2C(O)=C2C(=O)[C@]3(O)C(O)=C(C(N)=O)C(=O)[C@@H](N(C)C)[C@@H]3[C@@H](O)[C@H]12] | (4S,4aR,5S,5aR,12aR)- 4-(dimethylamino)- 1,5,10,11,12a- pentahydroxy-6- methylene-3,12-dioxo- 3,4,4a,5,5a,6,12,12a- octahydrotetracene-2- carboxamide (methacycline; rondomycin; CAS RN | | | | 82- 85% |
| | | 914-00-1; PubChem | | | | |
| | | CID 54675785) | | | | |
| 11 | 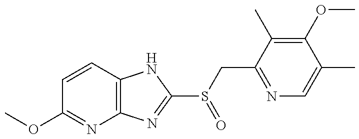 | 5-methoxy-2-(((4- methoxy-3,5- dimethylpyridin-2- yl)methyl)sulfinyl)-1H- imidazo[4,5-b]pyridine- (tenatoprazole; CAS RN 113712-98-4; PubChem CID 636411) | | | | 52- 63% |
| 12 | 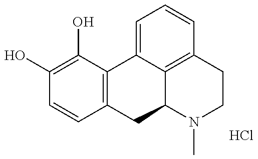 [SMILES: CN1CCc2cccc3c2[C@@H]1Cc1ccc(O)c(O)c1-3 ; Cl] | (S)-6-methyl-5,6,6a,7- tetrahydro-4H- dibenzo[de,g]quinoline- 10,11-diol hydrochloride ((S)-apomorphine hydrochloride; PubChem CID 6852389) | | | Ki = 1.61 | ~−95% |
| 13 | 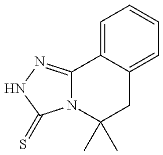 [SMILES: CC1(C)Cc2ccccc2-c2n[nH]c(=S)n21] | 5,5-dimethyl-5,6- dihydro- [1,2,4]triazolo[3,4- a]isoquinoline-3(2H)- thione (PubChem CID 870599) | | | Ki = 0.496 | ~−95% |
| 14 | 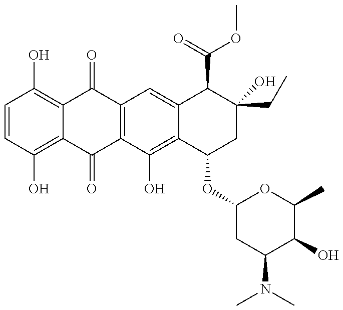 [SMILES: CC[C@@]1(O)C[C@H](O[C@H]2C[C@H](N(C)C)[C@H](O)[C@H](C)O2)c2c(cc3c(c2O)C(=O)c2c(O)ccc(O)c2C3=O)[C@H]1C(=O)OC] | methyl (1R,2R,4S)-4- (((2R,4S,5S,6S)-4- (dimethylamino)-5- hydroxy-6- methyltetrahydro-2H- pyran-2-yl)oxy)-2-ethyl- 2,5,7,10-tetrahydroxy- 6,11-dioxo- 1,2,3,4,6,11- hexahydrotetracene-1- carboxylate (Pyrromycin; CAS RN 668-17-7; PubChem CID 196990) | 0.73 | −67.06 | | |
| 15 | 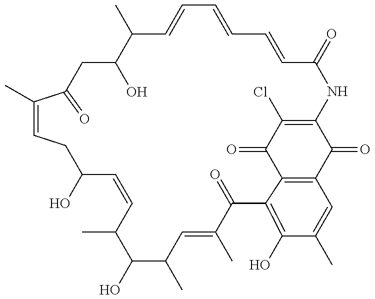 [SMILES: C/C1=C/CC(O)/C=C\C(C)C(O)C(C)/C=C(\C)C(=O)c2c(O)c(C)cc3c2C(=O)C(Cl)=C(NC(=O)/C=C/C=C/C=C/C(C)C(O)CC1=O)C3=O] | 2-chloro-13,19,23,28- tetrahydroxy- 12,16,22,24,26,29- hexamethyl- 13,14,19,22,23,24- hexahydro-1H-3,31- methanobenzo[e][1]aza- cyclononacosine- 1,5,15,27,32(4H,12H,18H)- pentaone (naphthomycin B; CAS RN 86825-88-9; PubChem CID 73698824) | 4.71 | −54.29 | | |
| 16 | 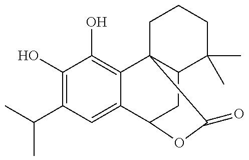 [SMILES: CC(C)c1cc2c(c(O)c1O)C13CCCC(C)(C)C1CC2OC3=O] | 5,6-dihydroxy-7- isopropyl-1,1-dimethyl- 1,3,4,9,10,10a- hexahydro-2H-9,4a- (epoxymethano)phenan- thren-12-one (CAS RN 5957-80-2; PubChem CID 2579) | | | 6.89 | −88.12 |
| 17 | 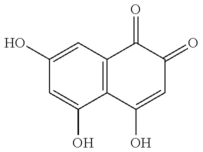 [SMILES: O=C1C=C(O)c2c(O)cc(O)cc2C1=O] | 4,5,7- trihydroxynaphthalene- 1,2-dione (flaviolin; CAS RN 479- 05-0; PubChem CID 160478) | | | 8.29 | −87.76 |
| 18 | 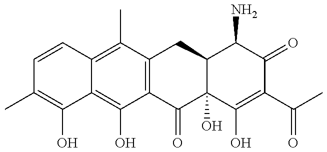 [SMILES: CC(=O)C1=C(O)[C@@]2(O)C(=O)c3c(c(C)c4ccc(C)c(O)c4c3O)C[C@H]2[C@@H](N)C1=O] | (1R,4aR,12aS)-3- acetyl-1-amino- 4,4a,6,7-tetrahydroxy- 8,11-dimethyl-12,12a- dihydrotetracene- 2,5(1H,4aH)-dione (cetocycline; CAS RN 29144-42-1; PubChem CID 71402) | | | 8.86 | −98.3 |
| 19 | 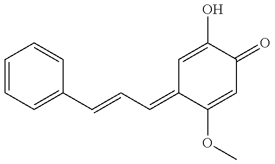 [SMILES: COC1=CC(=O)C(O)=C/C1=C\C=C\c1ccccc1] | (E)-2-hydroxy-5- methoxy-4-((E)-3- phenylallylidene)cyclo- hexa-2,5-dien-1-one (obtusaquinone; CAS RN 21105-15-7; PubChem CID 5967872) | | | 7.73 | −91.17 |
| 20 | 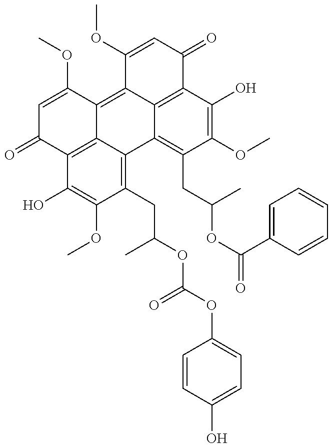 [SMILES: COc1c(O)c2c(=O)cc(OC)c3c4c(OC)cc(=O)c5c(O)c(OC)c(CC(C)OC(=O)c6ccccc6)c(c(c1CC(C)OC(=O)Oc1ccc(O)cc1)c23)c54] | 1-(3,10-dihydroxy-12- (2-(((4- hydroxyphenoxy)carbo- nyl)oxy)propyl)- 2,6,7,11-tetramethoxy- 4,9-dioxo-4,9- dihydroperylen-1- yl)propan-2-yl benzoate (calphostin C; CAS RN 121263-19-2; PubChem CID 2533) | | | 0.51 | −109.9 |
| 21 | 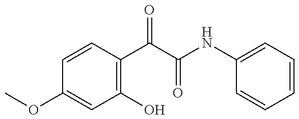 [SMILES: COc1ccc(C(=O)C(=O)Nc2ccccc2)c(O)c1] | 2-(2-hydroxy-4- methoxyphenyl)-2-oxo- N-phenylacetamide (PubChem CID 5082617) | | | 0.94 | −62.96 |
| 22 | 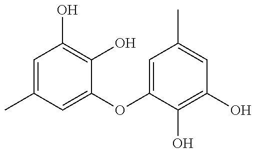 [SMILES: Cc1cc(O)c(O)c(Oc2cc(C)cc(O)c2O)c1] | 6,6′-oxybis(4- methylbenzene-1,2- diol) (Violaceol I; ethericin A; CAS RN 68027-81- 6; PubChem CID 100615) | | | 0.77 | −82.01 |
| 23 | 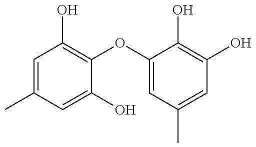 [SMILES: Cc1cc(O)c(Oc2cc(C)cc(O)c2O)c(O)c1] | 3-(2,6-dihydroxy-4- methylphenoxy)-5- methylbenzene-1,2- diol (Violaceol II; CAS RN 81827-49-8; PubChem CID 16196968) | | | 0.89 | −81.48 |
| 24 | 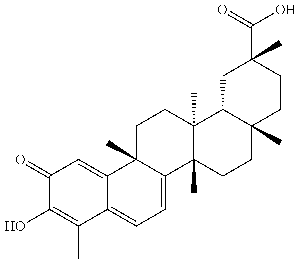 [SMILES: CC1=C(O)C(=O)C=C2C1=CC=C1[C@@]2(C)CC[C@@]2(C)[C@@H]3C[C@](C)(C(=O)O)CC[C@]3(C)CC[C@]12C] | (2R,4aS,6aS,12bR,14aS, 14bR)-10-hydroxy- 2,4a,6a,9,12b,14a- hexamethyl-11-oxo- 1,2,3,4,4a,5,6,6a,11,12b, 13,14,14a,14b- tetradecahydropicene- 2-carboxylic acid (celastrol; tripterin; CAS RN 34157-83-0; PubChem CID 122724) | | | 0.66 | −85.24 |
| 25 | 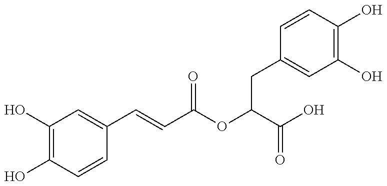 [SMILES: O=C(/C=C/c1ccc(O)c(O)c1)OC(Cc1ccc(O)c(O)c1)C(=O)O] | (E)-3-(3,4- dihydroxyphenyl)-2-((3- (3,4- dihydroxyphenyl)acryloyl) oxy)propanoic acid (rosmarinic acid; CAS RN 537-15-5; PubChem CID 5315615) | | | 3.22 | −90.27 |
| 26 | 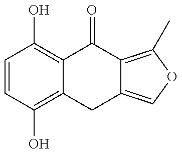 [SMILES: Cc1occ2c1C(=O)c1c(O)ccc(O)c1C2] | 5,8-dihydroxy-3- methylnaphtho[2,3- c]furan-4(9H)-one (MS-444; CAS RN 150045-18-4; PubChem CID 132904) | | | 0.69 | −87.06 |
| 27 | 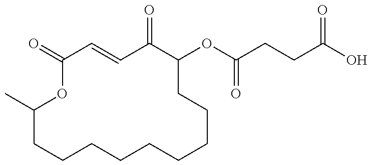 [SMILES: CC1CCCCCCCCCC(OC(=O)CCC(=O)O)C(=O)/C=C/C(=O)O1] | (E)-4-((16-methyl-2,5- dioxooxacyclohexadec- 3-en-6-yl)oxy)-4- oxobutanoic acid (PubChem CID 41880) | | | 3.85 | −83.95 |
| 28 | 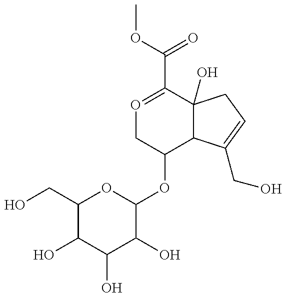 | methyl 4a-hydroxy-7- (hydroxymethyl)-1- ((3,4,5-trihydroxy-6- (hydroxymethyl)tetrahy- dro-2H-pyran-2- yl)oxy)-1,4a,5,7a- tetrahydrocyclopenta[c] pyran-4-carboxylate (PubChem CID 12443420) | | | 0.39 | −96.21 |
| 29 | 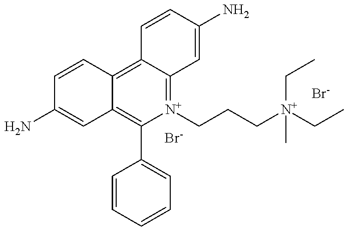 [SMILES: CC[N+](C)(CC)CCC[n+]1c(-c2ccccc2)c2cc(N)ccc2c2ccc(N)cc21 ; [Br-] ; [Br-]] | 3.8-diamino-5-(3- (diethyl(methyl)ammon- io)propyl)-6- phenylphenanthridin-5- ium bromide (propidium bromide; CAS RN 72460-87-8; PubChem CID 16043037) | | | 9.41 | −96.64 |
| 30 | 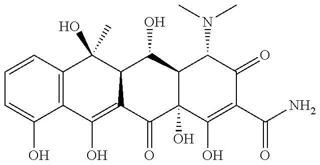 [SMILES: CN(C)[C@@H]1C(=O)C(C(N)=O)=C(O)[C@@]2(O)C(=O)C3=C(O)c4c(O)cccc4[C@@](C)(O)[C@H]3[C@H](O)[C@@H]12] | (4S,4aR,5S,5aR,6S,12aR)- 4-(dimethylamino)- 1,5,6,10,11,12a- hexahydroxy-6-methyl- 3,12-dioxo- 3,4,4a,5,5a,6,12,12a- octahydrotetracene-2- carboxamide (oxytetracycline; terramycin; CAS RN | | | 6.02 | −83.01 |
| | | 79-57-2; PubChem | | | | |
| | | CID 54675779) | | | | |
| 31 | 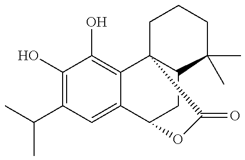 | (4aR,9S,10aS)-5,6- dihydroxy-7-isopropyl- 1,1-dimethyl- 1,3,4,9,10,10a- hexahydro-2H-9,4a- (epoxymethano)phenan- thren-12-one (carnosol; CAS RN 5957-80-2; PubChem CID 442009) | | | 5.37 | −78.21 |
| 32 | 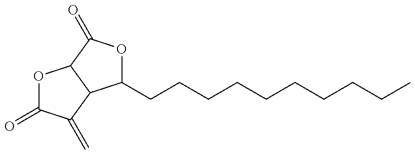 [SMILES: C=C1C(=O)OC2C(=O)OC(CCCCCCCCCC)C12] | 4-decyl-3- methylenedihydrofuro[3,4- b]furan- 2,6(3H,4H)-dione (PubChem CID 21125955) | | | 5.22 | −77.91 |
| 33 | 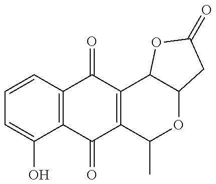 [SMILES: CC1OC2CC(=O)OC2C2=C1C(=O)c1c(O)cccc1C2=O] | 7-hydroxy-5-methyl- 3,3a,5,11b-tetrahydro- 2H-benzo[g]furo[3,2- c]isochromene-2,6,11- trione (kalafungin; CAS RN 29394-61-4; PubChem CID 283138) | | | 8.02 | −86.55 |
| 34 | 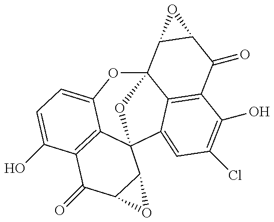 [SMILES: O=C1c2c(O)c(Cl)cc3c2[C@@]2(Oc4ccc(O)c5c4[C@]3(O2)[C@@H]2O[C@@H]2C5=O)[C@@H]2O[C@H]12] | (1aR,1bS,5aS,6aR,6bS, 11aS)-3-chloro-4,10- dihydroxy- 1a,5a,6a,11a- tetrahydro-5H,11H- 1b,6b- epoxyoxireno[2′,3′:2,3] naphtho[1,8- bc]oxireno[2′,3′:2,3]naph- tho[1,8-ef]oxepine- 5,11-dione (spiroxin A; PubChem CID 10070299) | | | 2.99 | −86.04 |
| 35 | 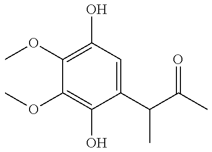 [SMILES: COc1c(O)cc(C(C)C(C)=O)c(O)c1OC] | 3-(2,5-dihydroxy-3,4- dimethoxyphenyl)butan- 2-one (PubChem CID 11195806) | | | 1.17 | −80.52 |

### Examples 36-41

The following compounds of Table 2 were obtained from commercial sources, and evaluated in fluorescence polarization (FP) competition assay.

Specifically, dose-dependent CALP inhibition of compounds of examples 36-41 was assessed by FP competition assays. Experiments were conducted similarly to HTS assays described above but in large-volume format. Briefly, CALP was expressed and purified as previously described (Amacher et al., 2011). A fluorescently labeled peptide, NPG32, was commercially obtained (sequence: F*-ANSRWQTSII where F* denotes a N-terminal fluorescein group coupled via an aminohexanoic acid linker). CALP was dialyzed into FP storage buffer (25 mM Tris pH 8.5, 150 mM NaCl, 0.1 mM TCEP, and 0.02% sodium azide). CALP was diluted into FP buffer (FP storage buffer supplemented with 0.1 mg/mL IgG and 30 μM thesit) containing 31.58 nM NPG32. The protein/reporter solution was aliquoted into 384-well plates with each well containing 19 μL of the mixture. Twelve-point titrations (2-fold serial dilutions) of the test compounds were prepared in 25% DMSO. To each well, 1 μL of compound was added. The final 20 μL solution contained: 1.854 PM CALP, 30 nM NPG32, 1.25% DMSO, and variable concentrations of the test compounds (maximal concentration ranged between 100-500 μM). Each well was mixed by pipette before plates were centrifuged for 3 min at 750×g. Plates were incubated at room temperature for 30 min before anisotropy was measured. Individual experiments were performed with triplicate technical replicates. Except for Examples 37-38, the reported potency for all compounds was calculated by averaging three experimental replicates. Assays containing Examples 37-38 were only conducted with one experimental replicate to determine absence of binding.

TABLE 2

| Ex. | | |
|---|---|---|
| No. | Chemical structure | Chemical name |
| 36 | 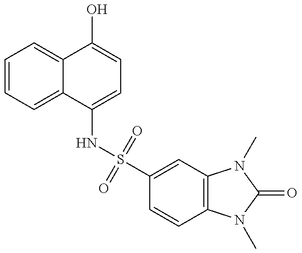 [SMILES: Cn1c(=O)n(C)c2cc(S(=O)(=O)Nc3ccc(O)c4ccccc34)ccc21] | N-(4-hydroxynaphthalen-1-yl)-1,3-dimethyl-2- oxo-2,3-dihydro-1H-benzo[d]imidazole-5- sulfonamide |
| 37 | 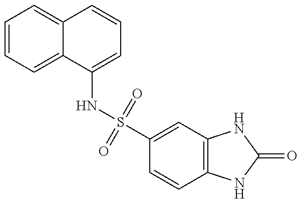 [SMILES: O=c1[nH]c2ccc(S(=O)(=O)Nc3cccc4ccccc34)cc2[nH]1] | N-(naphthalen-1-yl)-2-oxo-2,3-dihydro-1H- benzo[d]imidazole-5-sulfonamide |
| 38 | 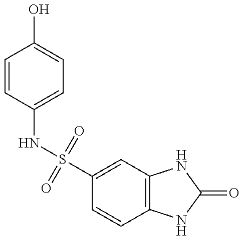 [SMILES: O=c1[nH]c2ccc(S(=O)(=O)Nc3ccc(O)cc3)cc2[nH]1] | N-(4-hydroxyphenyl)-2-oxo-2,3-dihydro-1H- benzo[d]imidazole-5-sulfonamide |
| 39 | 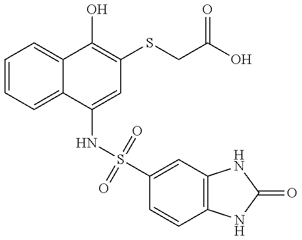 [SMILES: O=C(O)CSc1cc(NS(=O)(=O)c2ccc3[nH]c(=O)[nH]c3c2)c2ccccc2c1O] | 2-((1-hydroxy-4-((2-oxo-2,3-dihydro-1H- benzo[d]imidazole)-5- sulfonamido)naphthalen-2-yl)thio)acetic acid |
| 40 | 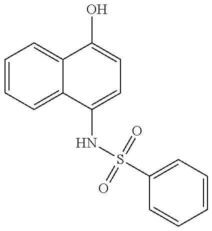 [SMILES: O=S(=O)(Nc1ccc(O)c2ccccc12)c1ccccc1] | N-(4-hydroxynaphthalen-1- yl)benzenesulfonamide |
| 41 | 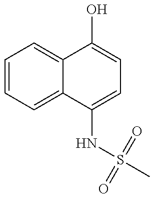 [SMILES: CS(=O)(=O)Nc1ccc(O)c2ccccc12] | N-(4-hydroxynaphthalen-1- yl)methanesulfonamide |

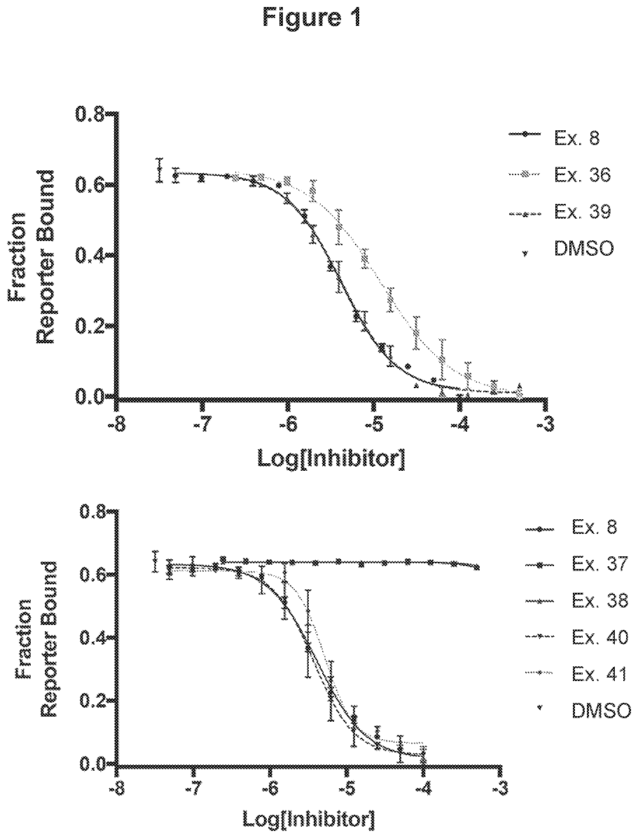

Examples 36-41 were evaluated for CALP inhibition using the above-provided procedure. FIG. 1 provides dose-response curves of these compounds as compared to treatment with DMSO (control) and example 8.

### Example 42: Evaluation of Example 8

Example 8 was found to covalently modify CAL PDZ by matrix-assisted laser desorption/ionization-time of flight (MALDI-TOF) spectrometry, using known procedures such as those described in Zhao et al. (ACS Appl. Mater. Interfaces 2018, 10, 43, 37732-37742). Following incubation of 50 μM protein with 75 μM of example 8, the molecular weight of the samples was determined by MALDI (unlabeled CALP molecular weight: 9464±15 Da; labeled CALP molecular weight: 9623±9 Da). Although example 8 is 355 Da, only a ˜160 Da adduct was observed for CAL PDZ. When the same reaction is performed with the point mutant CAL PDZC319A (where the only cysteine residue is mutated to alanine), no adduct is detected, indicating that example 8 covalently modifies CAL PDZ's cysteine residue.

It is hypothesized that cysteine, when deprotonated, acts as a nucleophile (Nu) and attacks the napthol group on 8, causing hydrolysis of the sulfonamide group. Without being bound to a theory, it is believed that the proposed adduct that remains covalently bound to CALP is:

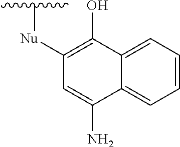

Interestingly, all analogs tested resulted in the same adduct size, suggesting that new chemical substituents also serve as leaving groups in the reaction.

Next, to probe the biochemical effect example 8 exerts on CAL PDZ-peptide binding, CAL PDZ was pre-labeled with example 8 and performed fluorescence polarization (FP) assays to determine apparent K_{D} values for both unlabeled and labeled CAL PDZ. Compared to unlabeled CAL PDZ, the adducted form (CAL PDZ—example 8) shows about a 4-fold attenuation in peptide binding: K_{D} of 1.03 μM vs. 4.38 μM. These data suggest that example 8 might acts as a covalent allosteric inhibitor of CAL PDZ.

¹H-¹⁵-N HSQC NMR measurements were performed using isotopically (¹⁵N) labeled CAL PDZ pre-treated with example 8 or DMSO. The control showed tight and non-overlapping peaks indicative of a well-folded protein. When CAL PDZ is adducted with example 8, some peaks show loss of signal intensity and show peak broadening, which is generally observed when a protein is unfolded. Because CAL PDZ—example 8 still engages in peptide binding via FP assays and remains soluble by size-exclusion chromatography, without being bound by a theory, it is believed that these data suggest that example 8 partially unfolds CAL PDZ but allows it to remain binding competent. It is hypothesized that when CAL PDZ is partially unfolded by example 8, the entropy cost of peptide binding increases, which in turn modulates its binding affinity.

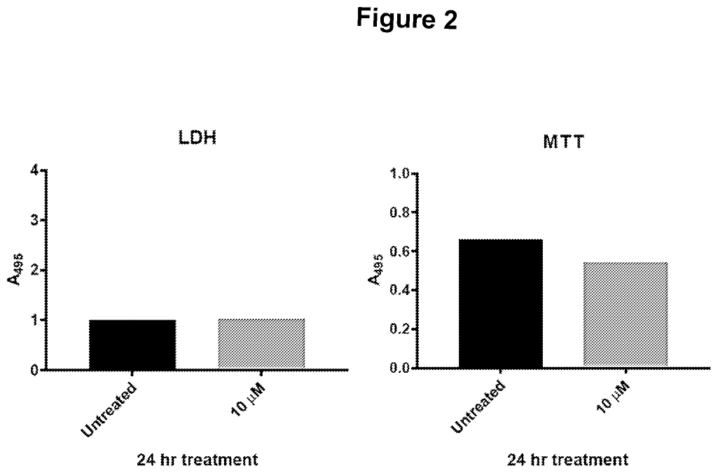

The effect of example 8 on cytotoxicity and anti-proliferation in wild-type CFBE cells was assessed. Upon addition of 10 μM compound, cells were found to contain similar levels of cellular toxicity and proliferation as untreated cells as provided in FIG. 2 using MTT cell viability assay (Thermo Fisher Scientific, Waltham, MA, USA) and CyQUANT™ LDH cytotoxicity assay (Thermo Fisher Scientific, Waltham, MA, USA).

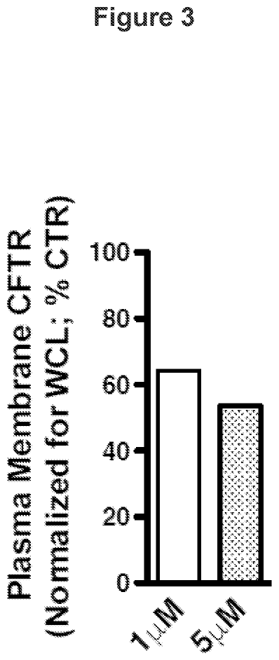

The effect of example 8 on cell-surface expression of wild-type CFTR was also tested in the absence of correctors or potentiators. Two doses (1 μM and 5 μM) of example 8 were applied to wild-type cystic fibrosis bronchial epithelial (WT-CFBE) cells and the abundance of WT-CFTR cells in the plasma membrane was quantified. As provided in FIG. 3, Example 8 was found to reduce surface levels of WT-CFTR.

### Example 43: Evaluation of Example 7

Example 7 was found to covalently modified CAL PDZ. Following incubation with the small molecule, there was observed a ˜300 Da shift by MALDI, corresponding to the example 7's molecular weight of 214 Da. Like example 8, example 7 attenuated CAL PDZ's ability to bind peptides by -4-fold: K_{D} of 1.03 μM vs. 4.29 μM.

Example 7 was applied to wild-type cystic fibrosis bronchial epithelial (WT-CFBE) cells in dosages of 5 μM, 10 μM, and 20 μM, and the change in short-circuit current was quantified. The cells were treated for 4 hours with example 7 or the DMSO control in FBS negative medium. Cells were fed with FBS negative medium 24 hours before treatment. Example 7 showed no significant stimulation in current activity at the three doses tested.

### Example 44: Evaluation of Example 12 and Example 13

200 nM Dab2-DH was equilibrated with fluorescently labeled peptide (F*-STA02, 30 nM) for 30 minutes at RT. Unlabeled competitor peptide (STA02) was serially diluted and mixed with the protein-reporter mix. Percent DMSO in all samples was 1%. Plates were mixed by vibration, centrifuged at 1200 g for 2 minutes and allowed to equilibrate at RT for 30-45 minutes before measurement. Fluorescence polarization was measured at an excitation wavelength of 470±5 nm and an emission wavelength of 525±20 nm.

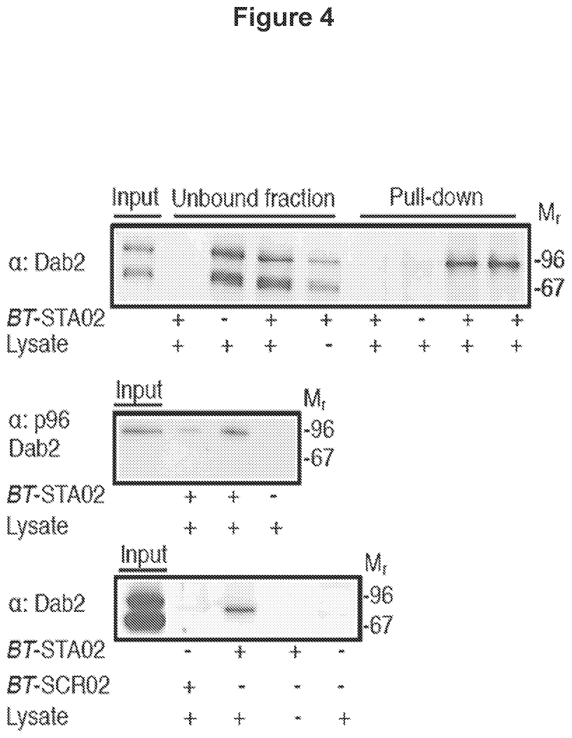

Fluorescent anisotropy measurements of 3 nM fluorescently labeled reported peptide, F*-STA02, at various protein concentrations revealed a K_{D}=63±10 nM. FP competitive displacement experiments revealed a STA02 Kᵢ=150±36 nM. ITC binding isotherms fit using a single-site binding model for STA02 peptide titration into a Dab2-DH solution in sodium phosphate buffer yielded a ΔH=−20.6 kcal/mol and K_{D}=151 nM. As shown in FIG. 4, immunoblots of input, supernatant, or pull-down fractions following detection with an anti-Dab2 antibody that interacts with either isoform (αDab2) or specifically with the p96 isoform (α-p96).

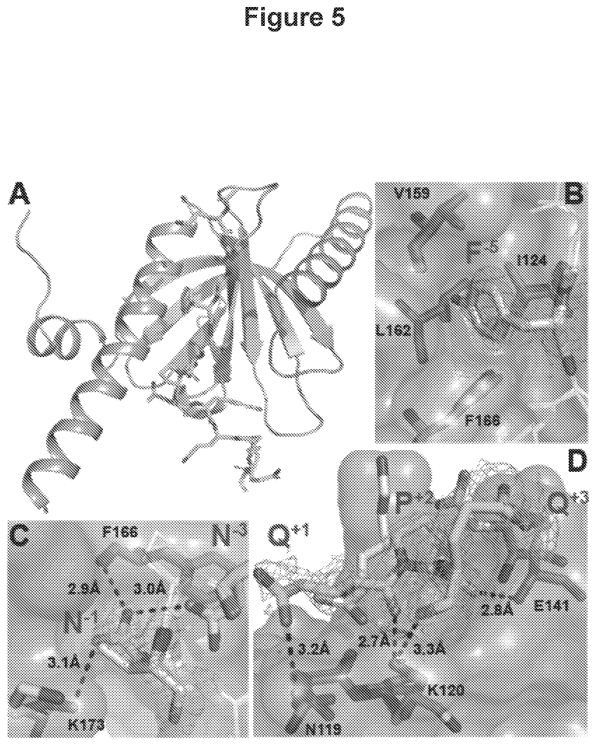

The crystal structure of the Dab2-DH:STA02 complex was resolved. FIG. 5, panel A shows the structure of the Dab2-DH domain and STA02 (shown as sticks). STA02 docks in the canonical peptide-binding cleft of the domain. Panels B, C, and D show interactions of individual affinity-enhancing STA02 substitutions with residues of the Dab2-DH binding pocket, along with electron density from the (2Fo-Fc) map as a mesh, calculated before inclusion of the peptide in the phasing model.

A high-throughput screen was conducted with positive (STA02) and negative (DMSO) controls. Positive hits were selected for further analysis. 2-Hydroxyestradiol, example 12, and example 13—were incubated for 2 hours with an equilibrated solution of BT-STA02:Dab2-DH:streptavidin beads. The beads were washed thrice with wash buffer and the bound protein was eluted with excess STA02 peptide. Eluted samples were examined by immunoblotting for Dab2-DH protein, FP competitive displacement experiments revealed a Ki=2.38±1.92 μM for 2-hydroxyestradiol, 1.61±0.74 μM for example 12, and 496±322 μM for example 13.

The toxicity and anti-proliterative effects of the compounds were investigated. In short, CFBE cells were seeded at 5×10⁸ in 96 well plates. Cells were treated in phenol- and fetal bovine serum (FBS)-free minimum essential medium (MEM, Gibco). WVT-CFBE cells grown in 96 well plates were treated with Triton X-100 (positive control), 0.2% DMSO (negative control) or 20 μM of the test compound for 24 or 48 hours. Assays were performed and measured according to the CytoTox 96 Non-Radioactive cytotoxicity Assay (G1780) kit (Promega) and the CellTiter 96 Aqueous One solution Cell proliferation Assay kit (Promega) instructions. Percent maximum toxicity (max. tox.) for the LDH assay was calculated.

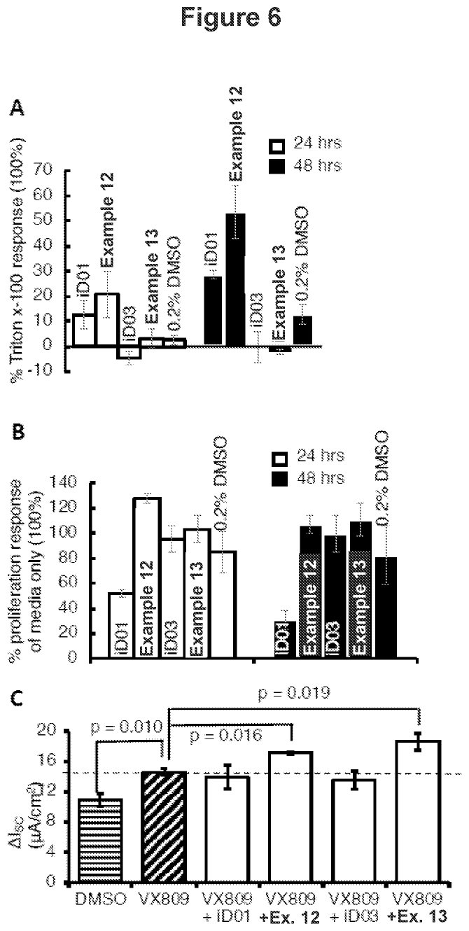

FIG. 6, panels A and B, show cytotoxicity and percent proliferation. Polarized CFBE-ΔF508 monolayers were exposed basolaterally for 48 hours to VS-809 (01 μM) and 2-hydroxyestradiol (identified as iD01), example 12, dipyridamole (identified as iD03), and example 13 at a concentration of 20 μM. Short-circuit current are expressed as the change (ΔI_{sc}) induced by CFTR inhibitor 172 (20 μM) and Kalydeco (10 μM).

### Example 45: Evaluation of Example 10

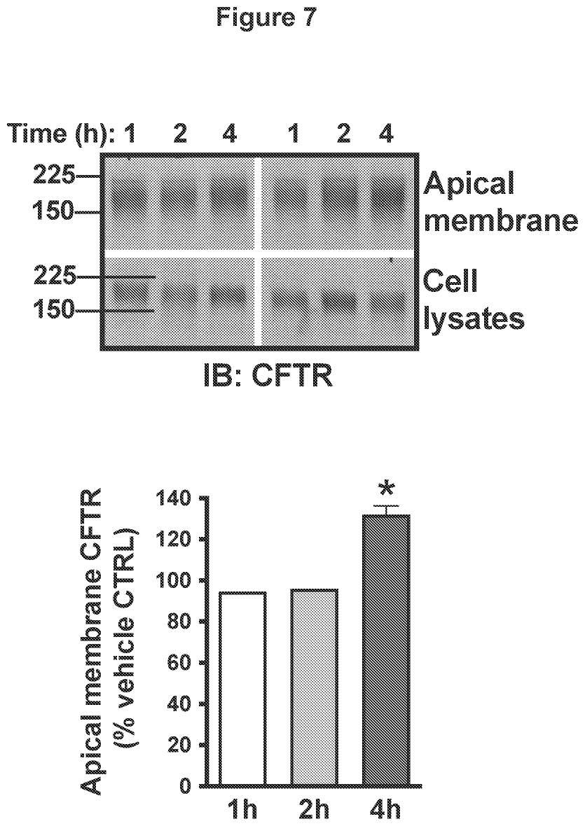

An increase in the abundance of CFTR was observed when WT-CFBE cells were treated with 1 μM example 10, as shown in FIG. 7. Representative immunoblots (top) and summary of biotinylation experiments (bottom) demonstrating effects of P2 on the apical membrane abundance of CFTR in WT-CFBE cells. The lowest concentration (1 μM) that showed an effect was used (data not shown). Apical membrane CFTR was normalized for CFTR abundance in cell lysate. N=2 (1h and 2 h) and n=3 (4h). Error bar S.E.M. *p<0.05 versus vehicle control (CTRL; 0.03% water).

A significant enhancement of CFTR activity was also observed when F508del HBE primary cells were treated with 1 μM of example 10 for 4 hours in conjunction with VX-770, Fsk, and IBMX. These data underscore the relative unresponsiveness of WT-CFBE cells to potential CFTR modulators. Further, significant enhancement of CFTR activity when F508del HBE primary cells were treated with 1 μM of example 10 for 4 hours in conjunction with VX-770, C-18, CFFT-002, Fsk, and IBMX. The observed channel activity was approximately 2-fold more active than when tested in the absence of C-18 and CFFT-002.

These results illustrate the importance of testing putative CFTR modulators in the context of primary cells and in combination with other CFTR modulators.

### Example 46: Evaluation of Example 6 and Example 9

A functional test for CFTR activity is the determination of airway surface liquid (ASL) height, reflecting ion transport and associated osmotic recruitment of water, following activation of CFTR ion transport via forskolin stimulation. This functional test is directly related to one of the hallmarks of CF: the viscosity of airway mucus due to lack of anion transport.

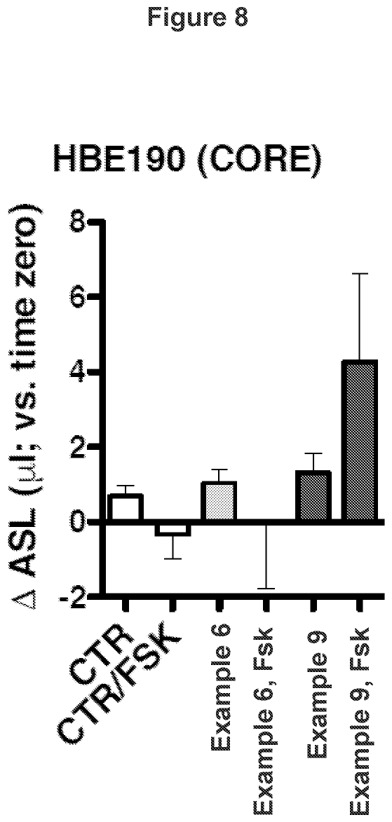

Example 6 or Example 9 (20 μM each) or DMSO (CTR; 0.02%; 1 L/5 mL) was added to the basolateral medium for 4 hr. Subsequently, forskolin (20 μM) and IBMX (1 mM) was added to the apical and basolateral medium. The test compounds were added for 30 min directly to the basolateral medium, and they were added in 10 μL medium to the apical side. The 10 μL volume was subtracted from the ASL volume (N=2-4/group). Example 9 increased the ASL volume generated in response to a forskolin stimulus when tested in HBE primary cells. See FIG. 8.

Some embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating cystic fibrosis, the method comprising administering to a subject in need of such treatment one or more compounds selected from:
(4S,4aR,5S,5aR,12aR)-4-(dimethylamino)-1,5,10,11,12a-pentahydroxy-6-methylene-3,12-dioxo-3,4,4a,5,5a,6,12,12a-octahydrotetracene-2-carboxamide (methacycline);
3,4′,5′-trihydroxy-5-methoxy-2′-methyl-[1,1′-biphenyl]-2-carboxylic acid (alutenusin);
(S)-6-methyl-5,6,6a,7-tetrahydro-4H-dibenzo[de,g]quinoline-10,11-diol hydrochloride ((S)-apomorphine hydrochloride);
5,5-dimethyl-5,6-dihydro-[1,2,4]triazolo[3,4-a]isoquinoline-3(2H)-thione;
N-(4-hydroxynaphthalen-1-yl)-2-oxo-2,3-dihydro-1H-benzo[d]imidazole-5-sulfonamide;
(2R,3R)-5,7-dihydroxy-2-(3,4,5-trihydroxy-6-oxo-1-((2R,3R)-3,5,7-trihydroxychroman-2-yl)-6H-benzo[7]annulen-8-yl)chroman-3-yl 3,4,5-trihydroxybenzoate;
6-methyl-2a,2a1,3,4,4a,5,6,7,8a,12b-decahydro-2H-4a1,5-ethanofuro[4′,3′,2′:4,10]anthra[9,1-bc]oxepine-2,9,12-trione;
(1aR,1bS,5aS,6aR,6bS,11aS)-3,9-dichloro-4,10-dihydroxy-1a,5a,6a,11a-tetrahydro-5H,11H-1b,6b-epoxyoxireno[2′,3′:2,3]naphtho[1,8-bc]oxireno[2′,3′:2,3]naphtho[1,8-ef]oxepine-5,11-dione;
3,4,5-trihydroxy-6-methylphthalaldehyde;
4-(4-(pyrimidin-2-yl)Piperazin-1-yl)Phenol;
(E)-5-chloro-9-(3-hydroxy-2-methylbutanoyl)-6a-methyl-3-(3-methylpent-1-en-1-yl)-6H-furo[2,3-h]isochromene-6,8(6aH)-dione;
1,8,9-trihydroxy-3-methoxy-6H-benzofuro[3,2-c]chromen-6-one;
3,4′,5′-trihydroxy-5-methoxy-2′-methyl-[1,1′-biphenyl]-2-carboxylic acid;
5-methoxy-2-(((4-methoxy-3,5-dimethylpyridin-2-yl)methyl)sulfinyl)-1H-imidazo[4,5-b]pyridine;
methyl (1R,2R,4S)-4-(((2R,4S,5S,6S)-4-(dimethylamino)-5-hydroxy-6-methyltetrahydro-2H-pyran-2-yl)oxy)-2-ethyl-2,5,7,10-tetrahydroxy-6,11-dioxo-1,2,3,4,6,11-hexahydrotetracene-1-carboxylate;
2-chloro-13,19,23,28-tetrahydroxy-12,16,22,24,26,29-hexamethyl-13,14,19,22,23,24-hexahydro-1H-3,31-methanobenzo[e][1]azacyclononacosine-1,5,15,27,32(4H,12H,18H)-pentaone;
5,6-dihydroxy-7-isopropyl-1,1-dimethyl-1,3,4,9,10,10a-hexahydro-2H-9,4a-(epoxymethano)phenanthren-12-one;
4,5,7-trihydroxynaphthalene-1,2-dione;
(1R,4aR,12aS)-3-acetyl-1-amino-4,4a,6,7-tetrahydroxy-8,11-dimethyl-12,12a-dihydrotetracene-2,5(1H,4aH)-dione;
(E)-2-hydroxy-5-methoxy-4-((E)-3-phenylallylidene)cyclohexa-2,5-dien-1-one;
1-(3,10-dihydroxy-12-(2-(((4-hydroxyphenoxy)carbonyl)oxy)propyl)-2,6,7,11-tetramethoxy-4,9-dioxo-4,9-dihydroperylen-1-yl)propan-2-yl benzoate;
2-(2-hydroxy-4-methoxyphenyl)-2-oxo-N-phenylacetamide;
6,6′-oxybis(4-methylbenzene-1,2-diol);
3-(2,6-dihydroxy-4-methylphenoxy)-5-methylbenzene-1,2-diol;
(2R,4aS,6aS,12bR,14aS,14bR)-10-hydroxy-2,4a,6a,9,12b,14a-hexamethyl-11-oxo-1,2,3,4,4a,5,6,6a,11,12b,13,14,14a,14b-tetradecahydropicene-2-carboxylic acid;
(E)-3-(3,4-dihydroxyphenyl)-2-((3-(3,4-dihydroxyphenyl)acryloyl)oxy)propanoic acid;
5,8-dihydroxy-3-methylnaphtho[2,3-c]furan-4(9H)-one;
(E)-4-((16-methyl-2,5-dioxooxacyclohexadec-3-en-6-yl)oxy)-4-oxobutanoic acid;
methyl 4a-hydroxy-7-(hydroxymethyl)-1-((3,4,5-tri hydroxy-6-(hydroxymethyl)tetrahydro-2H-pyran-2-yl)oxy)-1,4a,5,7a-tetrahydrocyclopenta[c]pyran-4-carboxylate;
3,8-diamino-5-(3-(diethyl(methyl)ammonio)propyl)-6-phenylphenanthridin-5-ium bromide;
(4S,4aR,5S,5aR,6S,12aR)-4-(dimethylamino)-1,5,6,10,11,12a-hexahydroxy-6-methyl-3,12-dioxo-3,4,4a,5,5a,6,12,12a-octahydrotetracene-2-carboxamide;
(4aR,9S,10aS)-5,6-dihydroxy-7-isopropyl-1,1-dimethyl-1,3,4,9,10,10a-hexahydro-2H-9,4a-(epoxymethano)phenanthren-12-one;
4-decyl-3-methylenedihydrofuro[3,4-b]furan-2,6(3H,4H)-dione;
7-hydroxy-5-methyl-3,3a,5,11b-tetrahydro-2H-benzo[g]furo[3,2-c]isochromene-2,6,11-trione;
(1aR,1bS,5aS,6aR,6bS,11aS)-3-chloro-4,10-dihydroxy-1a,5a,6a,11a-tetrahydro-5H,11H-1b,6b-epoxyoxireno[2′,3′:2,31naphtho[1,8-bc]oxireno[2′,3′:2,3]naphtho[1,8-ef]oxepine-5,11-dione;
3-(2,5-dihydroxy-3,4-dimethoxyphenyl)butan-2-one;
one or more compounds of formula (I): 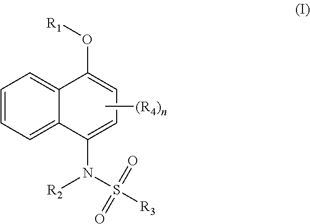
wherein,
n is an integer 0, 1, or 2;
R₁ is hydrogen or C₁-C₆ alkyl;
R₂ is hydrogen or C₁-C₆ alkyl;
R₃ is C₁-C₆ alkyl, aryl optionally substituted with one or more R₅, heteroaryl optionally substituted with one or more R₅, heterocyclyl optionally substituted with one or more R₅, or C₄-C₈ cycloalkyl optionally substituted with one or more R₅; and
R₄ is independently selected from halogen, —CN, —NO₂, C₁-C₆ alkyl optionally substituted with one or more R₅, C₁-C₆ haloalkyl, —NH₂, —NH(C₁-C₆ alkyl), —N(C₁-C₆ alkyl)₂, —OH, —O(C₁-C₆ alkyl optionally substituted with one or more R), C₁-C₆ haloalkoxy, —CONH₂, —CONH(C₁-C₆ alkyl), —CON(C₁-C₆ alkyl)₂, —CONH—OH, —CO₂H, —CO₂(C₁-C₆ alkyl), —SH, —S(C₁-C₆ alkyl optionally substituted with one or more R), —SO₂R₇, —SO₂OR₇, —SO₂N(R₇)₂, aryl optionally substituted with one or more R₆, heteroaryl optionally substituted with one or more R₆, heterocyclyl optionally substituted with one or more R₆, and C₃-C₈ cycloalkyl optionally substituted with one or more R;
wherein
each R₅ is independently selected from the group consisting of halogen, —NO₂, —CN, C₁-C₆ alkyl, C₁-C₆ haloalkyl, —OH, C₁-C₆ alkoxy, C₁-C₆ haloalkoxy, —CONH₂, —CONH(C₁-C₆ alkyl), —CON(C₁-C₆ alkyl)₂, —CO₂H, —CO₂(C₁-C₆ alkyl), —SO₂R₇, —SO₂OR₇, and —SO₂N(R₇)₂;
each R₆ is independently selected from the group consisting of halogen, —NO₂, —CN, C₁-C₆ alkyl, C₁-C₆ haloalkyl, —OH, C₁-C₆ alkoxy, and C₁-C₆ haloalkoxy; and
each R₇ is independently selected from the group consisting of hydrogen, C₁-C₆ alkyl, phenyl, or tolyl; and
pharmaceutically acceptable salts thereof.

2-29. (canceled)

30. The method of claim 1, wherein the compound is administered as a pharmaceutical composition comprising the compound and a pharmaceutically acceptable carrier, solvent, adjuvant or diluent.

31. The method of claim 1, further comprising administering a secondary therapeutic agent.

32. The method of claim 31, wherein the secondary therapeutic agent is a CFTR modulator.

33. The method of claim 31, wherein the secondary therapeutic agent is selected from one or more of antibiotics, anti-inflammatory agents, mucoactive agents, and combinations thereof.

34. A method of inhibiting protein interactions with PDZ domain of the cystic fibrosis transmembrane conductance regulator (CFTR)-associated ligand (CAL), the method comprising administering to a subject in need of such treatment one or more compounds recited in claim 1, optionally in combination with a secondary therapeutic agent.

35. A method of inhibiting protein interactions with DH domain of Disabled-2 (Dab2) protein, the method comprising administering to a subject in need of such treatment one or more compounds recited in claim 1, optionally in combination with a secondary therapeutic agent.
